SEQUENCE LISTING (51 sequences)

SEQ ID NO: 1 - protein, 727 aa, Clostridium phage
MDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDVEEPKTFHIVQNNNIVYGCNVYMDKFFEVENKDGSTTEIGLCLDFFDSYKIITCAESQFHNYVHDVDPMFYKMGEEYDYDTWRSPTHKQTTLELRYQYNDIYMLREVIEQFYIDGLCGGELPLTGMRTASSIAFNVLKKMTFGEEKTEEGYINYFELDKKTKFEFLRKRIEMESYTGGYTHANHKAVGKTINKIGCSLDINSSYPSQMAYKVFPYGKPVRKTWGRKPKTEKNEVYLIEVGFDFVEPKHEEYALDIFKIGAVNSKALSPITGAVSGQEYFCTNIKDGKAIPVYKELKDTKLTTNYNVVLTSVEYEFWIKHFNFGVFKKDEYDCFEVDNLEFTGLKIGSILYYKAEKGKFKPYVDHFTKMKVENKKLGNKPLTNQAKLILNGAYGKFGTKQNKEEKDLIMDKNGLLTFTGSVTEYEGKEFYRPYASFVTAYGRLQLWNAIIYAVGVENFLYCDTDSIYCNREVNSLIEDMNAIGETIDKTILGKWDVEHVFDKFKVLGQKKYMYHDCKEDKTDLKCCGLPSDARKIIIGQGFDEFYLGKNVEGKKQRKKVIGGCLLLDTLFTIKKIMF

SEQ ID NO: 2 - protein, 739 aa, synthetic construct
  REGION 1..739: note = His tagged wild type Pol6
MHHHHHHHHSGGSDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDVEEPKTFHIVQNNNIVYGCNVYMDKFFEVENKDGSTTEIGLCLDFFDSYKIITCAESQFHNYVHDVDPMFYKMGEEYDYDTWRSPTHKQTTLELRYQYNDIYMLREVIEQFYIDGLCGGELPLTGMRTASSIAFNVLKKMTFGEEKTEEGYINYFELDKKTKFEFLRKRIEMESYTGGYTHANHKAVGKTINKIGCSLDINSSYPSQMAYKVFPYGKPVRKTWGRKPKTEKNEVYLIEVGFDFVEPKHEEYALDIFKIGAVNSKALSPITGAVSGQEYFCTNIKDGKAIPVYKELKDTKLTTNYNVVLTSVEYEFWIKHFNFGVFKKDEYDCFEVDNLEFTGLKIGSILYYKAEKGKFKPYVDHFTKMKVENKKLGNKPLTNQAKLILNGAYGKFGTKQNKEEKDLIMDKNGLLTFTGSVTEYEGKEFYRPYASFVTAYGRLQLWNAIIYAVGVENFLYCDTDSIYCNREVNSLIEDMNAIGETIDKTILGKWDVEHVFDKFKVLGQKKYMYHDCKEDKTDLKCCGLPSDARKIIIGQGFDEFYLGKNVEGKKQRKKVIGGCLLLDTLFTIKKIMF

SEQ ID NO: 3 - protein, 739 aa, synthetic construct
  REGION 1..739: note = Pol6-P1743
MHHHHHHHHSGGSDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDKEEPKTFHIVQNNNIVYGCNVYMDKFFEVENKDGSTTEIGLCLDFFDSYKIITCAESKFHNLVHDVDPMFYKMGEEYDADTWRSPTHKQTTLELRYQYNDIYMLREVIEQFYIDGLCGGELPLTGMRTASSIAFNRLKKMTFGEEKTEEGYINYFELDKKTKFEFLRKRIEMESYTGGYTHANHKAVGKTINKIGCSLDINSAYPSQMAYKVFPYGKPVRKTWGRKPKTEKNEVYLIEVGFDFVEPKHEEYALDIFKIGAVNSKALSPITGAVSGQEYFCTNIKDGKAIPVYKELKDTKLTTNYNVVLTSVEYEFWIKHFNFGVFKKDEYDCFEVDNLEFTGLKIGSILYYKAEKGKFKFYVDHYMKMKVRNKKLGNRPLALQFKLILNGAYGKFGTGQNKEEKDLIMDKNGLLTFTGSVTKYEGKEFYRPYASFVTAYGRLQLWNAIIYAVGVENFLYCDTDSIYCNREVRSLIEDMNAIGETIDKTILGKWRVEHVFDKFKVLGQKKYMYHDCKQDYTDLKCCGLPYDARKIIIGQGFDEFYLGKNVEGKKQRKKVIGGCLLLDTLFTIKKIMF

SEQ ID NO: 4 - protein, 739 aa, synthetic construct
  REGION 1..739: note = Pol6-P2094
MHHHHHHHHSGGSDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDKEEPKTFHIVQNNNIVYGCNVYMDKFFEVENKDGSTTEIGLCLDFFDSYKIITCAESKFHNLVHDVDPMFYKMGEEYDADTWRSPTHKQTTLELRYQYNDIYMLREVIEQFYIDGLCGGELPLTGMRTASSIAFNRLKKMTFGEEKTEEGYINYFELDKKTKFEFLRKRIEMESYTGGYTHANHKAVGKTINKIGCSLDINSAYPSQMAYKVFPYGKPVRKTWGRKPKTEKNEVYLIEVGFDFVEPKHEEYALDIFKIGAVNSKALSPITGAVSGQEYFCTNIKDGKAIPVYKELKDTKLTTNYNVVLTSVEYEFWIKHFNFGVFKKDEYDCFEVDNLEFTGLKIGSILYYKAEKGKFKFYVIHYMKMKVRNKKLGNRPLALQFKLILNGAYGKFGTGQNKEEKDLIMDKNGLLTFTGSVTKYEGKEFYRPYASFVTAYGRLQLWNAIIYAVGVENFLYCDTDSIYCNREVRSLIEDMNAIGETIDKTILGKWRVEHVFDKFKVLGQKKYMYHDCKQDYTDLKCCGLPYDARKIIIGQGFDEFYLGKNVEGKKQRKKVIGGCLLLDTLFTIKKIMF

SEQ ID NO: 5 - protein, 739 aa, synthetic construct
  REGION 1..739: note = Pol6-P2271
MHHHHHHHHSGGSDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDKEEPKTFHIVQNNNIVYGCNVYMDKFFEVEWKDGSTTEIGLCLDFFDSYKIITCAESKFHNLVHDVDPMFYKMGEEYDADTWRSPTHKQTTLELRYQYNDIYMLREVIEQFYIDGLCGGELPLTGMRTASSIAFNRLKKMTFGEEKTEEGYINYFELDKKTKFEFLRKRIEMESYTGGYTHANHKAVGKTINKIGCSLDINSAYPSQMAYKVFPYGKPVRKTWGRKPKTEKNEVYLIEVGFDFVEPKHEEYALDIFKIGAVNSKALSPITGAVSGQEYFCTNIKDGKAIPVYKELKDTKLTTNYNVVLTSVEYEFWIKHFNFGVFKKDEYDCFEVDNLEFTGLKIGSILYYKAEKGKFKFYVDHYMKMKVRNKKLGNRPLALQFKLILNGAYGKFGTGQNKEEKDLIMDKNGLLTFTGSVTKYEGKEFYRPYASFVTAYGRLQLWNAIIYAVGVENFLYCDTDSIYCNREVRSLIEDMNAIGETIDKTILGKWRVEHVFDKFKVLGQKKYMYHDCKQDYTDLKCCGLPYDARKIIIGQGFDEFYLGKNVEGKKQRKKVIGGCLLLDTLFTIKKIMF

SEQ ID NO: 6 - protein, 739 aa, synthetic construct
  REGION 1..739: note = Pol6-P2303
MHHHHHHHHSGGSDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDKEEPKTFHIVQNNNIVYGCNVYMDKFFEVENKDGSTTEIGLCLDFFDSYKIITCAESKFHNLVHDVDPMFYKMGEEYDADTWRSPTHKQTTLELRYQYNDIYMLREVIEQFYIDGLCGGELPLTGMRTASSIAFNRLKKMTFGEEKTEEGYINYFELDKKTKFEFLRKRIEMESYHGGYTHANHKAVGKTINKIGCSLDINSAYPSQMAYKVFPYGKPVRKTWGRKPKTEKNEVYLIEVGFDFVEPKHEEYALDIFKIGAVNSKALSPITGAVSGQEYFCTNIKDGKAIPVYKELKDTKLTTNYNVVLTSVEYEFWIKHFNFGVFKKDEYDCFEVDNLEFTGLKIGSILYYKAEKGKFKFYVDHYMKMKVRNKKLGNRPLALQFKLILNGAYGKFGTGQNKEEKDLIMDKNGLLTFTGSVTKYEGKEFYRPYASFVTAYGRLQLWNAIIYAVGVENFLYCDTDSIYCNREVRSLIEDMNAIGETIDKTILGKWRVEHVFDKFKVLGQKKYMYHDCKQDYTDLKCCGLPYDARKIIIGQGFDEFYLGKNVEGKKQRKKVIGGCLLLDTLFTIKKIMF

SEQ ID NO: 7 - protein, 739 aa, synthetic construct
  REGION 1..739: note = Pol6-P2546
MHHHHHHHHSGGSDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDKEEPKTFHIVQNNNIVYGCNVYMDKFFEVEWKDGSTTEIGLCLDFFDSYKIITCAESKFHNLVHDVDPMFYKMGEEYDVDTWRSPTHKQTTLELRYQYNDIYMLREVIEQFYIDGLCGGELPLTGMRTASSIAFNRLKKMTFGEEKTEEGYINYFELDKKTKFEFLRKRIEMESYTGGYTHANHKAVGKTINKIGCSLDINSAYPSQMAYKVFPYGKPVRKTWGRKPKTEKNEVYLIEVGFDFVEPKHEEYALDIFKIGAVNSKALSPITGAVSGQEYFCTNIKDGKAIPVYKELKDTKLTTNYNVVLTSVEYEFWIKHFNFGVFKKDEYDCFEVDNLEFTGLKIGSILYYKAEKGKFKFYVDHYMKMKVRNKKLGNRPLALQFKLILNGAYGKFGTGQNKEEKDLIMDKNGLLTFTGSVTKYEGKEFYRPYASFVTAYGRLQLWNAIIYAVGVENFLYCDTDSIYCNREVRSLIEDMNAIGETIDKTILGKWRVEHVFDKFKVLGQKKYMYHDCKQDYTDLKCCGLPYDARKIIIGQGFDEFYLGKNVEGKKQRKKVIGGCLLLDTLFTIKKIMF

SEQ ID NO: 8 - protein, 739 aa, synthetic construct
  REGION 1..739: note = Pol6-P2569
MHHHHHHHHSGGSDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDKEEPKTFHIVQNNNIVYGCNVYMDKFFEVEWKDGSTTEIGLCLDFFDSYKIITCAESKFHNLVHDVDPMFYKMGEEYDADTWRSPTHKQTTLELRYQYNDIYMLREVIEQFYIDGLCGGELPLTGMRTASSIAFNRLKKMTFGEEKTEEGYINYFELDKKTKFEFLRKRIEMESYTGGYTHANHKAVGKTINKIGCSLDINSAYPSQMAYKVFPYGKPVRKTWGRKPKTEKNEVYLIEVGFDFVEPKHEEYALDIFKIGAVNSKALSPITGAVSGQEYFCTNIKDGKAIPVYKELKDTKLTTNYNVVLTSVEYEFWIKHFNFGVFKKDEYDCFEVDNLEFTGLKIGSILYYKAEKGKFKFYVDHYMKMKVRNKKLGNRPLALQFKLILNGAYGKFGTGQNKEEKDLIMDKNGLLTFTGSVTKYEGKEFYRPYASFVTAYGRLQLWNAIIYAVGVENFLYCDTDSIYCNREVRSLIEDMNAIGETIDKTILGGWRVEHVFDKFKVLGQKKYMYHDCKQDYTDLKCCGLPYDARKIIIGQGFDEFYLGKNVEGKKQRKKVIGGCLLLDTLFTIKKIMF

SEQ ID NO: 9 - protein, 739 aa, synthetic construct
  REGION 1..739: note = Pol6-P2570
MHHHHHHHHSGGSDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDKEEPKTFHIVQNNNIVYGCNVYMDKFFEVEWKDGSTTEIGLCLDFFDSYKIITCAESKFHNLVHDVDPMFYKMGEEYDADTWRSPTHKQTTLELRYQYNDIYMLREVIEQFYIDGLCGGELPLTGMRTASSIAFNRLKKMTFGEEKTEEGYINYFELDKKTKFEFLRKRIEMESYTGGYTHANHKAVGKTINKIGCSLDINSAYPSQMAYKVFPYGKPVRKTWGRKPKTEKNEVYLIEVGFDFVEPKHEEYALDIFKIGAVNSKALSPITGAVSGQEYFCTNIKDGKAIPVYKELKDTKLTTNYNVVLTSVEYEFWIKHFNFGVFKKDEYDCFEVDNLEFTGLKIGSILYYKAEKGKFKFYVDHYMKMKVRNKKLGNRPLALQFKLILNGAYGKFGTGQNKEEKDLIMDKLGLLTFTGSVTKYEGKEFYRPYASFVTAYGRLQLWNAIIYAVGVENFLYCDTDSIYCNREVRSLIEDMNAIGETIDKTILGKWRVEHVFDKFKVLGQKKYMYHDCKQDYTDLKCCGLPYDARKIIIGQGFDEFYLGKNVEGKKQRKKVIGGCLLLDTLFTIKKIMF

SEQ ID NO: 10 - protein, 739 aa, synthetic construct
  REGION 1..739: note = Pol6-P2571
MHHHHHHHHSGGSDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDKEEPKTFHIVQNNNIVYGCNVYMDKFFEVEWKDGSTTEIGLCLDFFDSYKIITCAESKFHNLVHDVDPMFYKMGEEYDADTWRSPTHKQTTLELRYQYNDIYMLREVIEQFYIDGLCGGELPLTGMRTASSIAFNRLKKMTFGEEKTEEGYINYFELDKKTKFEFLRKRIEMESYTGGYTHANHKAVGKTINKIGCSLDINSAYPSQMAYKVFPYGKPVRKTWGRKPKTEKNEVYLIEVGFDFVEPKHEEYALDIFKIGAVNSKALSPITGAVSGQEYFCTNIKDGKAIPVYKELKDTKLTTNYNVVLTSVEYEFWIKHFNFGVFKKDEYDCFEVDNLEFTGLKIGSILYYKAEKGKFKFYVDHYMKMKVRNKKLGNRPLALQFKLILNGAYGKFGTGQNKEEKDLIMDKNGLLTFTGSVTKYEGKEFYRPYASFVTAYGRLQLWNAIIYAVGVENFLYCDTDSIYCNREVRFLIEDMNAIGETIDKTILGKWRVEHVFDKFKVLGQKKYMYHDCKQDYTDLKCCGLPYDARKIIIGQGFDEFYLGKNVEGKKQRKKVIGGCLLLDTLFTIKKIMF

SEQ ID NO: 11 - protein, 739 aa, synthetic construct
  REGION 1..739: note = Pol6-P2573
MHHHHHHHHSGGSDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDKEEPKTFHIVQNNNIVYGCNVYMDKFFEVEWKDGSTTEIGLCLDFFDSYKIITCAESKFHNLVHDVDPMFYKMGEEYDVDTWRSPTHKQTTLELRYQYNDIYMLREVIEQFYIDGLCGGELPLTGMRTASSIAFNRLKKMTFGEEKTEEGYINYFELDKKTKFEFLRKRIEMESYTGGYTHANHKAVGKTINKIGCSLDINSAYPSQMAYKVFPYGKPVRKTWGRKPKTEKNEVYLIEVGFDFVEPKHEEYALDIFKIGAVNSKALSPITGAVSGQEYFCTNIKDGKAIPVYKELKDTKLTTNYNVVLTSVEYEFWIKHFNFGVFKKDEYDCFEVDNLEFTGLKIGSILYYKAEKGKFKFYVDHYMKMKVRNKKRGNRPLALQFKLILNGAYGKFGTGQNKEEKDLIMDKNGLLTFTGSVTKYEGKEFYRPYASFVTAYGRLQLWNAIIYAVGVENFLYCDTDSIYCNREVRSLIEDMNAIGETIDKTILGKWRVEHVFDKFKVLGQKKYMYHDCKQDYTDLKCCGLPYDARKIIIGQGFDEFYLGKNVEGKKQRKKVIGGCLLLDTLFTIKKIMF

SEQ ID NO: 12 - protein, 739 aa, synthetic construct
  REGION 1..739: note = Pol6-P2579
MHHHHHHHHSGGSDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDKEEPKTFHIVQNNNIVYGCNVYMDKFFEVEWKDGSTTEIGLCLDFFDSYKIITCAESKFHNLVHDVDPMFYKMGEEYDVDTWRSPTHKQTTLELRYQYNDIYMLREVIEQFYIDGLCGGELPLTGMRTASSIAFNRLKKMTFGEEKTEEGYINYFELDKKTKFEFLRKRIEMESYTGGYTHANHKAVGKTINKIGCSLDINSAYPSQMAYKVFPYGKPVRKTWGRKPKTEKNEVYLIEVGFDFVEPKHEEYALDIFKIGAVNSKALSPITGAVSGQEYFCTNIKDGKAIPVYKELKDTKLTTNYNVVLTSVEYEFWIKHFNFGVFKKDEYDCFEVDNLEFTGLKIGSILYYKAEKGKFKFYVDHYMKMKVRNKKLGNRPLALQFKLILNGAYGKFGTGQNKEEKDLIMDKNGLLTFTGSVTKYEGKEFYRPYASFVTAYGRLQLWNAIIYAVGVENFLYCDTDSIYCNRFVRSLIEDMNAIGETIDKTILGKWRVEHVFDKFKVLGQKKYMYHDCKQDYTDLKCCGLPYDARKIIIGQGFDEFYLGKNVEGKKQRKKVIGGCLLLDTLFTIKKIMF

SEQ ID NO: 13 - protein, 831 aa, synthetic construct
  REGION 1..831: note = Pol6-P2271-SpyCatcher
MHHHHHHHHSGSATHIKFSKRDEDGKELAGATMELRDSSGKTISTWISDGQVKDFYLYPGKYTFVETAAPDGYEVATAITFTVNEQGQVTVNGKATKGDAHIGGSDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDKEEPKTFHIVQNNNIVYGCNVYMDKFFEVEWKDGSTTEIGLCLDFFDSYKIITCAESKFHNLVHDVDPMFYKMGEEYDADTWRSPTHKQTTLELRYQYNDIYMLREVIEQFYIDGLCGGELPLTGMRTASSIAFNRLKKMTFGEEKTEEGYINYFELDKKTKFEFLRKRIEMESYTGGYTHANHKAVGKTINKIGCSLDINSAYPSQMAYKVFPYGKPVRKTWGRKPKTEKNEVYLIEVGFDFVEPKHEEYALDIFKIGAVNSKALSPITGAVSGQEYFCTNIKDGKAIPVYKELKDTKLTTNYNVVLTSVEYEFWIKHFNFGVFKKDEYDCFEVDNLEFTGLKIGSILYYKAEKGKFKFYVDHYMKMKVRNKKLGNRPLALQFKLILNGAYGKFGTGQNKEEKDLIMDKNGLLTFTGSVTKYEGKEFYRPYASFVTAYGRLQLWNAIIYAVGVENFLYCDTDSIYCNREVRSLIEDMNAIGETIDKTILGKWRVEHVFDKFKVLGQKKYMYHDCKQDYTDLKCCGLPYDARKIIIGQGFDEFYLGKNVEGKKQRKKVIGGCLLLDTLFTIKKIMF

SEQ ID NO: 14 - protein, 720 aa, synthetic construct
  REGION 1..720: note = P2268
MHHHHHHHHSGWSDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDKEEPKTFHIVQNNNIVYGCNVYMDKFFEVENKDGSTTEIGLCLDFFDSYKIITCAESKFHNLVHDVDPMFYKMGEEYDADTWRSPTHKQTTLELRYQYNDIYMLREVIEQFYIDGLCGGELPLTGMRTASSIAFNRLKKMTFGEEKTEEGYINYFELDKKTKFEFLRKRIEMESYTGGYTHANHKAVGKTINKIGCSLDINSAYPSQMAYKVFPYGKPVRKTWGRKPKTEKNEVYLIEVGFDFVEPKHEEYALDIFKIGAVNSKALSPITGAVSGQEYFCTNIKDGKAIPVYKELKDTKLTTNYNVVLTSVEYEFWIKHFNFGVFKKDEYDCFEVDNLEFTGLKIGSILYYKAEKGKFKFYVDHYMKMKVRNKKLGNRPLALQFKLILNGAYGKFGTGQNKEEKDLIMDKNGLLTFTGSVTKYEGKEFYRPYASFVTAYGRLQLWNAIIYAVGVENFLYCDTDSIYCNREVRSLIEDMNAIGETIDKTILGKWRVEHVFDKFKVLGQKKYMYHDCKQDYTDLKCCGLPYDARKIIIGQGFDEFYLGKNVEGKKQRKK

SEQ ID NO: 15 - protein, 720 aa, synthetic construct
  REGION 1..720: note = 2269
MHHHHHHHHSGGSDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDKEEPKTFHIVQNNNIVYGCNVYMDKFFEVENKDGSTTEIGLCLDFFDSYKIITCAESKFHNLVHDVDPMFYKMGEEYDADTWRSPTHKQTTLELRYQYNDIYMLREVIEQFYIDGLCGGELPLTGMRTASSIAFNRLKKMTFGEEKTEEGYINYFELDKKTKFEFLRKRIEMESYTGGYTHANHKAVGKTINKIGCSLDINSAYPSQMAYKVFPYGKPVRKTWGRKPKTEKNEVYLIEVGFDFVEPKHEEYALDIFKIGAVNSKALSPITGAVSGQEYFCTNIKDGKFIPVYKELKDTKLTTNYNVVLTSVEYEFWIKHFNFGVFKKDEYDCFEVDNLEFTGLKIGSILYYKAEKGKFKFYVDHYMKMKVRNKKLGNRPLALQFKLILNGAYGKFGTGQNKEEKDLIMDKNGLLTFTGSVTKYEGKEFYRPYASFVTAYGRLQLWNAIIYAVGVENFLYCDTDSIYCNREVRSLIEDMNAIGETIDKTILGKWRVEHVFDKFKVLGQKKYMYHDCKQDYTDLKCCGLPYDARKIIIGQGFDEFYLGKNVEGKKQRKK

SEQ ID NO: 16 - protein, 720 aa, synthetic construct
  REGION 1..720: note = 2270
MHHHHHHHHSGYSDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDKEEPKTFHIVQNNNIVYGCNVYMDKFFEVENKDGSTTEIGLCLDFFDSYKIITCAESKFHNLVHDVDPMFYKMGEEYDADTWRSPTHKQTTLELRYQYNDIYMLREVIEQFYIDGLCGGELPLTGMRTASSIAFNRLKKMTFGEEKTEEGYINYFELDKKTKFEFLRKRIEMESYTGGYTHANHKAVGKTINKIGCSLDINSAYPSQMAYKVFPYGKPVRKTWGRKPKTEKNEVYLIEVGFDFVEPKHEEYALDIFKIGAVNSKALSPITGAVSGQEYFCTNIKDGKAIPVYKELKDTKLTTNYNVVLTSVEYEFWIKHFNFGVFKKDEYDCFEVDNLEFTGLKIGSILYYKAEKGKFKFYVDHYMKMKVRNKKLGNRPLALQFKLILNGAYGKFGTGQNKEEKDLIMDKNGLLTFTGSVTKYEGKEFYRPYASFVTAYGRLQLWNAIIYAVGVENFLYCDTDSIYCNREVRSLIEDMNAIGETIDKTILGKWRVEHVFDKFKVLGQKKYMYHDCKQDYTDLKCCGLPYDARKIIIGQGFDEFYLGKNVEGKKQRKK

SEQ ID NO: 17 - protein, 720 aa, synthetic construct
  REGION 1..720: note = 2272
MHHHHHHHHSGGSDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDKEEPKTFHIVQNNNIVYGCNVYMDKFFEVENKDGSTTEIGLCLDFFDSYKIITCAESKFHNLVHDVDPMFYKMGEEYDADTWRSPTHKQTTLELRYQYNDIYMLREVIEQFYIDGLCGGELPLTGMRTASSIAFNRLKKMTFGEEKTEEGYINYFELDKKTKFEFLRKRIEMESYTGGYTHANHKAVGKTINKIGCSLDINSAYPSQMAYKVFPYGKPVRKTWGRKPKTEKNEVYLIEVGFDFVEPKHEEYALDIFKIGAVNSKALSPITGAVSGQEYFCTNIKDGKWIPVYKELKDTKLTTNYNVVLTSVEYEFWIKHFNFGVFKKDEYDCFEVDNLEFTGLKIGSILYYKAEKGKFKFYVDHYMKMKVRNKKLGNRPLALQFKLILNGAYGKFGTGQNKEEKDLIMDKNGLLTFTGSVTKYEGKEFYRPYASFVTAYGRLQLWNAIIYAVGVENFLYCDTDSIYCNREVRSLIEDMNAIGETIDKTILGKWRVEHVFDKFKVLGQKKYMYHDCKQDYTDLKCCGLPYDARKIIIGQGFDEFYLGKNVEGKKQRKK

SEQ ID NO: 18 - protein, 720 aa, synthetic construct
  REGION 1..720: note = 2273
MHHHHHHHHSGGSDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDKEEPKTFHIVQNNNIVYGCNVYMDKFFEVENKDGSTTEIGLCLDFFDSYKIITCAESKFHNLVHDVDPMFYKMGEEYDADTWRSPTHKQTTLELRYQYNDIYMLREVIEQFYIDGLCGGELPLTGMRTASSIAFNRLKKMTFGEEKTEEGYINYFELDKKTKFEFLRKRIEMESYTGGYTHANHKAVGKTINKIGCSLDINSAYPSQMAYKVFPYGKPVRKTWGRKPKTEKNEVYLIEVGFDFVEPKHEEYALDIFKIGAVNSKALSPITGAVSGQEYFCTNIKDGKAIPVYKELKDTKLTTNYNVVLTSVEYEFWIKHFNFGVFKKDEYDCFEVDNLEFTGLKIGSILYYKAEKGKFKFYVDHYMKMKVRNKKLGNRPLALQFKLILNGAYGKFGTGQNKIEKDLIMDKNGLLTFTGSVTKYEGKEFYRPYASFVTAYGRLQLWNAIIYAVGVENFLYCDTDSIYCNREVRSLIEDMNAIGETIDKTILGKWRVEHVFDKFKVLGQKKYMYHDCKQDYTDLKCCGLPYDARKIIIGQGFDEFYLGKNVEGKKQRKK

SEQ ID NO: 19 - protein, 720 aa, synthetic construct
  REGION 1..720: note = 2274
MHHHHHHHHSGFSDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDKEEPKTFHIVQNNNIVYGCNVYMDKFFEVENKDGSTTEIGLCLDFFDSYKIITCAESKFHNLVHDVDPMFYKMGEEYDADTWRSPTHKQTTLELRYQYNDIYMLREVIEQFYIDGLCGGELPLTGMRTASSIAFNRLKKMTFGEEKTEEGYINYFELDKKTKFEFLRKRIEMESYTGGYTHANHKAVGKTINKIGCSLDINSAYPSQMAYKVFPYGKPVRKTWGRKPKTEKNEVYLIEVGFDFVEPKHEEYALDIFKIGAVNSKALSPITGAVSGQEYFCTNIKDGKAIPVYKELKDTKLTTNYNVVLTSVEYEFWIKHFNFGVFKKDEYDCFEVDNLEFTGLKIGSILYYKAEKGKFKFYVDHYMKMKVRNKKLGNRPLALQFKLILNGAYGKFGTGQNKEEKDLIMDKNGLLTFTGSVTKYEGKEFYRPYASFVTAYGRLQLWNAIIYAVGVENFLYCDTDSIYCNREVRSLIEDMNAIGETIDKTILGKWRVEHVFDKFKVLGQKKYMYHDCKQDYTDLKCCGLPYDARKIIIGQGFDEFYLGKNVEGKKQRKK

SEQ ID NO: 20 - protein, 720 aa, synthetic construct
  REGION 1..720: note = 2275
MHHHHHHHHSGGSDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDKEEPKTFHIVQNNNIVYGCNVYMDKFFEVENKDGSTTEIGLCLDFFDSYKIITCAESKFHNLVHDVDPMFYKMGEEYDADTWRSPTHKQTTLELRYQYNDIYMLREVIEQFYIDGLCGGELPLTGMRTASSIAFNRLKKMTFGEEKTEEGYINYFELDKKTKFEFLRKRIEMESYTGGYTHANHKAVGKTINKIGCSLDINSAYPSQMAYKVFPYGKPVRKTWGRKPKTEKNEVYLIEVGFDFVEPKHEEYALDIFKIGAVNSKALSPITGAVSGQEYFCTNIKDGKAIPVYKELKDTKLTTNYNVVLTSVEYEFWIKHFNFGVFKKDEYDCFEVDNLEFTGLKIGSILYYKAEKGKFKFYVDHYMKMKVRNKKLGNRPLALQFKLILNGAYGKFGTGQNKEEKDLIMDKNGLLTFTGSVTKYEGKYFYRPYASFVTAYGRLQLWNAIIYAVGVENFLYCDTDSIYCNREVRSLIEDMNAIGETIDKTILGKWRVEHVFDKFKVLGQKKYMYHDCKQDYTDLKCCGLPYDARKIIIGQGFDEFYLGKNVEGKKQRKK

SEQ ID NO: 21 - protein, 720 aa, synthetic construct
  REGION 1..720: note = 2276
MHHHHHHHHSGGSDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDKEEPKTFHIVQNNNIVYGCNVYMDKFFEVENKDGSTTEIGLCLDFFDSYKIITCAESKFHNLVHDVDPMFYKMGEEYDADTWRSPTHKQTTLELRYQYNDIYMLREVIEQFYIDGLCGGELPLTGMRTASSIAFNRLKKMTFGEEKTEEGYINYFELDKKTKFEFLRKRIEMESYTGGYTHANHKAVGKTINKIGCSLDINSAYPSQMAYKVFPYGKPVRKTWGRKPKTEKNEVYLIEVGFDFVEPKHEEYALDIFKIGAVNSKALSPITGAVSGQEYFCTNIKDGKAIPVYKELKDTKLTTNYNVVLTSVEYEFWIKHFNFGVFKKDEYDCFEVDNLEFTGLKIGSILYYKAEKGKFKFYVDHYMKMKVRNKKLGNRPLALQFKLILNGAYGKFGTGQNKEEKDLIMDKNGLLTFTGSVTKYEGKEFYRPYASFVTAYGRLQLWNAIIYAVGVENFLYCDTDSIYCNREVRSLIEDMNAIGETIDKTILGKWRVEHVFDKFKVLGQKKYMYHDCKQHYTDLKCCGLPYDARKIIIGQGFDEFYLGKNVEGKKQRKK

SEQ ID NO: 22 - protein, 739 aa, synthetic construct
  REGION 1..739: note = 2564
MHHHHHHHHSGGSDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDKEEPKTFHIVQNNNIVYGCNVYMDKFFEVEWKDGSTTEIGLCLDFFDSYKIITCAESKFHNLVHDVDPMFYKMGEEYDADTWRSPTHKQTTLELRYQYNDIYMLREVIEQFYIDGLCGGELPLTGMRTASSIAFLRLKKMTFGEEKTEEGYINYFELDKKTKFEFLRKRIEMESYTGGYTHANHKAVGKTINKIGCSLDINSAYPSQMAYKVFPYGKPVRKTWGRKPKTEKNEVYLIEVGFDFVEPKHEEYALDIFKIGAVNSKALSPITGAVSGQEYFCTNIKDGKAIPVYKELKDTKLTTNYNVVLTSVEYEFWIKHFNFGVFKKDEYDCFEVDNLEFTGLKIGSILYYKAEKGKFKFYVDHYMKMKVRNKKLGNRPLALQFKLILNGAYGKFGTGQNKEEKDLIMDKNGLLTFTGSVTKYEGKEFYRPYASFVTAYGRLQLWNAIIYAVGVENFLYCDTDSIYCNREVRSLIEDMNAIGETIDKTILGKWRVEHVFDKFKVLGQKKYMYHDCKQDYTDLKCCGLPYDARKIIIGQGFDEFYLGKNVEGKKQRKKVIGGCLLLDTLFTIKKIMF

SEQ ID NO: 23 - protein, 739 aa, synthetic construct
  REGION 1..739: note = 2565
MHHHHHHHHSGGSDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDKEEPKTFHIVQNNNIVYGCNVYMDKFFEVEWKDGSTTEIGLCLDFFDSYKIITCAESKFHNLVHDVDPMFYKMGEEYDADTWRSPTHKQTTLELRYQYNDIYMLREVIEQFYIDGLCGGELPLTGMRTASSIAFNRLKKMTFGEEKTEEGYINYFELDKKTKFEFLRKRIEMESYTGGYTHANHKAVGKTINKIGCSLDINSAYPSQMAYKVFPYGKPVRKTWGRKPKTEKNEVYLIEVGFDFVEPKHEEYALDIFKIGAVNSKALSPITGAVSGQEYFCTNIKDGKAIPVYKELKDTKLTTNYNVVLTSVEYEFWIKHFNFGVFKKDEYDCFEVDNLEFTGLKIGSILYYKAEKGKFKFYVDHYMKMKVRNKKLGNRPLALQFKLILNGAYGKFGTGQNKEEKDLHMDKNGLLTFTGSVTKYEGKEFYRPYASFVTAYGRLQLWNAIIYAVGVENFLYCDTDSIYCNREVRSLIEDMNAIGETIDKTILGKWRVEHVFDKFKVLGQKKYMYHDCKQDYTDLKCCGLPYDARKIIIGQGFDEFYLGKNVEGKKQRKKVIGGCLLLDTLFTIKKIMF

SEQ ID NO: 24 - protein, 739 aa, synthetic construct
  REGION 1..739: note = 2566
MHHHHHHHHSGGSDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDKEEPKTFHIVQNNNIVYGCNVYMDKFFEVEWKDGSTTEIGLCLDFFDSYKIITCAESKFHNLVHDVDPMFYKMGEEYDADTWRSPTHKQTTLELRYQYNDIYMLREVIEQFYIDGLCGGELPLTGMRTASSIAFNRLKKMTFGEEKTEEGYINYFELDKKTKFEFLRKRIEMESYTGGYTHANHKAVGKTINKIGCSLDINSAYPSQMAYKVFPYGKPVRKTWGRKPKTEKNEVYLIEVGFDFVEPKHEEYALDIFKIGAVNSKALSPITGAVSGQEYFCTNIKDGKAIPVYKELKDTKLTTNYNVVLTSVEYEFWIKHFNFGVFKKDEYDCFEVDNLEFTGLKIGSILYYKAEKGKFKFYVDHYMKMKVRNKKLGNRPLALQFKLILNGAYGKFGTGQNKEEKDLTMDKNGLLTFTGSVTKYEGKEFYRPYASFVTAYGRLQLWNAIIYAVGVENFLYCDTDSIYCNREVRSLIEDMNAIGETIDKTILGKWRVEHVFDKFKVLGQKKYMYHDCKQDYTDLKCCGLPYDARKIIIGQGFDEFYLGKNVEGKKQRKKVIGGCLLLDTLFTIKKIMF

SEQ ID NO: 25 - protein, 739 aa, synthetic construct
  REGION 1..739: note = 2567
MHHHHHHHHSGGSDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDKEEPKTFHIVQNNNIVYGCNVYMDKFFEVEWKDGSTTEIGLCLDFFDSYKIITCAESKFHNLVHDVDPMFYKMGEEYDADTWRSPTHKQTTLELRYQYNDIYMLREVIEQFYIDGLCGGELPLTGMRTASSIAFNRLKKMTFGEEKTEEGYINYFELDKKTKFEFLRKRIEMESYTGGYTHANHKAVGKTINKIGCSLDINSAYPSQMAYKVFPYGKPVRKTWGRKPKTEKNEVYLIEVGFDFVEPKHEEYALDIFKIGAVNSKALSPITGAVSGQEYFCTNIKDGKAIPVYKELKDTKLTTNYNVVLTSVEYEFWIKHFNFGVFKKDEYDCFEVDNLEFTGLKIGSILYYKAEKGKFKFYVDHYMKMKVRNKKLGNRPLALQFKLILNGAYGKFGTGQNKEEKDLFMDKNGLLTFTGSVTKYEGKEFYRPYASFVTAYGRLQLWNAIIYAVGVENFLYCDTDSIYCNREVRSLIEDMNAIGETIDKTILGKWRVEHVFDKFKVLGQKKYMYHDCKQDYTDLKCCGLPYDARKIIIGQGFDEFYLGKNVEGKKQRKKVIGGCLLLDTLFTIKKIMF

SEQ ID NO: 26 - protein, 739 aa, synthetic construct
  REGION 1..739: note = 2568
MHHHHHHHHSGGSDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDKEEPKTFHIVQNNNIVYGCNVYMDKFFEVEWKDGSTTEIGLCLDFFDSYKIITCAESKFHNLVHDVDPMFYKMGEEYDADTWRSPTHKQTTLELRYQYNDIYMLREVIEQFYIDGLCGGELPLTGMRTASSIAFNRLKKMTFGEEKTEEGYINYFELDKKTKFEFLRKRIEMESYTGGYTHANHKAVGKTINKIGCSLDINSAYPSQMAYKVFPYGKPVRKTWGRKPKTEKNEVYLIEVGFDFVEPKHEEYALDIFKIGAVNSKALSPITGAVSGQEYFCTNIKDGKAIPVYKELKDTKLTTNYNVVLTSVEYEFWIKHFNFGVFKKDEYDCFEVDNLEFTGLKIGSILYYKAEKGKFKFYVDHYMKMKVRNKKLGNRPLALQFKLILNGAYGKFGTGQNKEEKDLIMDKNGLLTFTGSVTKYEGKEFYRPYASFVTAYGRLQLWNAIIYAVGVENFLYCDTDSIYCNRWVRSLIEDMNAIGETIDKTILGKWRVEHVFDKFKVLGQKKYMYHDCKQDYTDLKCCGLPYDARKIIIGQGFDEFYLGKNVEGKKQRKKVIGGCLLLDTLFTIKKIMF

SEQ ID NO: 27 - protein, 739 aa, synthetic construct; identical to SEQ ID NO: 8
  REGION 1..739: note = 2569

SEQ ID NO: 28 - protein, 739 aa, synthetic construct; identical to SEQ ID NO: 5
  REGION 1..739: note = 2570

SEQ ID NO: 29 - protein, 739 aa, synthetic construct; identical to SEQ ID NO: 10
  REGION 1..739: note = 2571

SEQ ID NO: 30 - protein, 739 aa, synthetic construct
  REGION 1..739: note = 2572
MHHHHHHHHSGGSDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDKEEPKTFHIVQNNNIVYGCNVYMDKFFEVEWKDGSTTEIGLCLDFFDSYKIITCAESKFHNLVHDVDPMFYKMGEEYDVDTWRSPTHKQTTLELRYQYNDIYMLREVIEQFYIDGLCGGELPLTGMRTASSIAFLRLKKMTFGEEKTEEGYINYFELDKKTKFEFLRKRIEMESYTGGYTHANHKAVGKTINKIGCSLDINSAYPSQMAYKVFPYGKPVRKTWGRKPKTEKNEVYLIEVGFDFVEPKHEEYALDIFKIGAVNSKALSPITGAVSGQEYFCTNIKDGKAIPVYKELKDTKLTTNYNVVLTSVEYEFWIKHFNFGVFKKDEYDCFEVDNLEFTGLKIGSILYYKAEKGKFKFYVDHYMKMKVRNKKLGNRPLALQFKLILNGAYGKFGTGQNKEEKDLIMDKNGLLTFTGSVTKYEGKEFYRPYASFVTAYGRLQLWNAIIYAVGVENFLYCDTDSIYCNREVRSLIEDMNAIGETIDKTILGKWRVEHVFDKFKVLGQKKYMYHDCKQDYTDLKCCGLPYDARKIIIGQGFDEFYLGKNVEGKKQRKKVIGGCLLLDTLFTIKKIMF

SEQ ID NO: 31 - protein, 739 aa, synthetic construct; identical to SEQ ID NO: 11
  REGION 1..739: note = 2573

SEQ ID NO: 32 - protein, 739 aa, synthetic construct
  REGION 1..739: note = 2574
MHHHHHHHHSGGSDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDKEEPKTFHIVQNNNIVYGCNVYMDKFFEVEWKDGSTTEIGLCLDFFDSYKIITCAESKFHNLVHDVDPMFYKMGEEYDVDTWRSPTHKQTTLELRYQYNDIYMLREVIEQFYIDGLCGGELPLTGMRTASSIAFNRLKKMTFGEEKTEEGYINYFELDKKTKFEFLRKRIEMESYTGGYTHANHKAVGKTINKIGCSLDINSAYPSQMAYKVFPYGKPVRKTWGRKPKTEKNEVYLIEVGFDFVEPKHEEYALDIFKIGAVNSKALSPITGAVSGQEYFCTNIKDGKAIPVYKELKDTKLTTNYNVVLTSVEYEFWIKHFNFGVFKKDEYDCFEVDNLEFTGLKIGSILYYKAEKGKFKFYVDHYMKMKVRNKKLGNRPLALQFKLILNGAYGKFGTGQNKEEKDLHMDKNGLLTFTGSVTKYEGKEFYRPYASFVTAYGRLQLWNAIIYAVGVENFLYCDTDSIYCNREVRSLIEDMNAIGETIDKTILGKWRVEHVFDKFKVLGQKKYMYHDCKQDYTDLKCCGLPYDARKIIIGQGFDEFYLGKNVEGKKQRKKVIGGCLLLDTLFTIKKIMF

SEQ ID NO: 33 - protein, 739 aa, synthetic construct
  REGION 1..739: note = 2575
MHHHHHHHHSGGSDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDKEEPKTFHIVQNNNIVYGCNVYMDKFFEVEWKDGSTTEIGLCLDFFDSYKIITCAESKFHNLVHDVDPMFYKMGEEYDVDTWRSPTHKQTTLELRYQYNDIYMLREVIEQFYIDGLCGGELPLTGMRTASSIAFNRLKKMTFGEEKTEEGYINYFELDKKTKFEFLRKRIEMESYTGGYTHANHKAVGKTINKIGCSLDINSAYPSQMAYKVFPYGKPVRKTWGRKPKTEKNEVYLIEVGFDFVEPKHEEYALDIFKIGAVNSKALSPITGAVSGQEYFCTNIKDGKAIPVYKELKDTKLTTNYNVVLTSVEYEFWIKHFNFGVFKKDEYDCFEVDNLEFTGLKIGSILYYKAEKGKFKFYVDHYMKMKVRNKKLGNRPLALQFKLILNGAYGKFGTGQNKEEKDLTMDKNGLLTFTGSVTKYEGKEFYRPYASFVTAYGRLQLWNAIIYAVGVENFLYCDTDSIYCNREVRSLIEDMNAIGETIDKTILGKWRVEHVFDKFKVLGQKKYMYHDCKQDYTDLKCCGLPYDARKIIIGQGFDEFYLGKNVEGKKQRKKVIGGCLLLDTLFTIKKIMF

SEQ ID NO: 34 - protein, 739 aa, synthetic construct
  REGION 1..739: note = 2576
MHHHHHHHHSGGSDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDKEEPKTFHIVQNNNIVYGCNVYMDKFFEVEWKDGSTTEIGLCLDFFDSYKIITCAESKFHNLVHDVDPMFYKMGEEYDVDTWRSPTHKQTTLELRYQYNDIYMLREVIEQFYIDGLCGGELPLTGMRTASSIAFNRLKKMTFGEEKTEEGYINYFELDKKTKFEFLRKRIEMESYTGGYTHANHKAVGKTINKIGCSLDINSAYPSQMAYKVFPYGKPVRKTWGRKPKTEKNEVYLIEVGFDFVEPKHEEYALDIFKIGAVNSKALSPITGAVSGQEYFCTNIKDGKAIPVYKELKDTKLTTNYNVVLTSVEYEFWIKHFNFGVFKKDEYDCFEVDNLEFTGLKIGSILYYKAEKGKFKFYVDHYMKMKVRNKKLGNRPLALQFKLILNGAYGKFGTGQNKEEKDLGMDKNGLLTFTGSVTKYEGKEFYRPYASFVTAYGRLQLWNAIIYAVGVENFLYCDTDSIYCNREVRSLIEDMNAIGETIDKTILGKWRVEHVFDKFKVLGQKKYMYHDCKQDYTDLKCCGLPYDARKIIIGQGFDEFYLGKNVEGKKQRKKVIGGCLLLDTLFTIKKIMF

SEQ ID NO: 35 - protein, 739 aa, synthetic construct
  REGION 1..739: note = 2577
MHHHHHHHHSGGSDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDKEEPKTFHIVQNNNIVYGCNVYMDKFFEVEWKDGSTTEIGLCLDFFDSYKIITCAESKFHNLVHDVDPMFYKMGEEYDVDTWRSPTHKQTTLELRYQYNDIYMLREVIEQFYIDGLCGGELPLTGMRTASSIAFNRLKKMTFGEEKTEEGYINYFELDKKTKFEFLRKRIEMESYTGGYTHANHKAVGKTINKIGCSLDINSAYPSQMAYKVFPYGKPVRKTWGRKPKTEKNEVYLIEVGFDFVEPKHEEYALDIFKIGAVNSKALSPITGAVSGQEYFCTNIKDGKAIPVYKELKDTKLTTNYNVVLTSVEYEFWIKHFNFGVFKKDEYDCFEVDNLEFTGLKIGSILYYKAEKGKFKFYVDHYMKMKVRNKKLGNRPLALQFKLILNGAYGKFGTGQNKEEKDLIMDKLGLLTFTGSVTKYEGKEFYRPYASFVTAYGRLQLWNAIIYAVGVENFLYCDTDSIYCNREVRSLIEDMNAIGETIDKTILGKWRVEHVFDKFKVLGQKKYMYHDCKQDYTDLKCCGLPYDARKIIIGQGFDEFYLGKNVEGKKQRKKVIGGCLLLDTLFTIKKIMF

SEQ ID NO: 36 - protein, 739 aa, synthetic construct
  REGION 1..739: note = 2578
MHHHHHHHHSGGSDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDKEEPKTFHIVQNNNIVYGCNVYMDKFFEVEWKDGSTTEIGLCLDFFDSYKIITCAESKFHNLVHDVDPMFYKMGEEYDVDTWRSPTHKQTTLELRYQYNDIYMLREVIEQFYIDGLCGGELPLTGMRTASSIAFNRLKKMTFGEEKTEEGYINYFELDKKTKFEFLRKRIEMESYTGGYTHANHKAVGKTINKIGCSLDINSAYPSQMAYKVFPYGKPVRKTWGRKPKTEKNEVYLIEVGFDFVEPKHEEYALDIFKIGAVNSKALSPITGAVSGQEYFCTNIKDGKAIPVYKELKDTKLTTNYNVVLTSVEYEFWIKHFNFGVFKKDEYDCFEVDNLEFTGLKIGSILYYKAEKGKFKFYVDHYMKMKVRNKKLGNRPLALQFKLILNGAYGKFGTGQNKEEKDLIMDKNGLLTFTGSVTKYEGKEFYRPYASFVTAYGRLQLWNAIIYAVGVENFLYCDTDSIYCNRWVRSLIEDMNAIGETIDKTILGKWRVEHVFDKFKVLGQKKYMYHDCKQDYTDLKCCGLPYDARKIIIGQGFDEFYLGKNVEGKKQRKKVIGGCLLLDTLFTIKKIMF

SEQ ID NO: 37 - protein, 739 aa, synthetic construct; identical to SEQ ID NO: 12
  REGION 1..739: note = 2579

SEQ ID NO: 38 - protein, 739 aa, synthetic construct
  REGION 1..739: note = 2580
MHHHHHHHHSGGSDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDKEEPKTFHIVQNNNIVYGCNVYMDKFFEVEWKDGSTTEIGLCLDFFDSYKIITCAESKFHNLVHDVDPMFYKMGEEYDVDTWRSPTHKQTTLELRYQYNDIYMLREVIEQFYIDGLCGGELPLTGMRTASSIAFNRLKKMTFGEEKTEEGYINYFELDKKTKFEFLRKRIEMESYTGGYTHANHKAVGKTINKIGCSLDINSAYPSQMAYKVFPYGKPVRKTWGRKPKTEKNEVYLIEVGFDFVEPKHEEYALDIFKIGAVNSKALSPITGAVSGQEYFCTNIKDGKAIPVYKELKDTKLTTNYNVVLTSVEYEFWIKHFNFGVFKKDEYDCFEVDNLEFTGLKIGSILYYKAEKGKFKFYVDHYMKMKVRNKKLGNRPLALQFKLILNGAYGKFGTGQNKEEKDLIMDKNGLLTFTGSVTKYEGKEFYRPYASFVTAYGRLQLWNAIIYAVGVENFLYCDTDSIYCNREVRFLIEDMNAIGETIDKTILGKWRVEHVFDKFKVLGQKKYMYHDCKQDYTDLKCCGLPYDARKIIIGQGFDEFYLGKNVEGKKQRKKVIGGCLLLDTLFTIKKIMF

SEQ ID NO: 39 - protein, 739 aa, synthetic construct
  REGION 1..739: note = 2581
MHHHHHHHHSGGSDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDKEEPKTFHIVQNNNIVYGCNVYMDKFFEVEWKDGSTTEIGLCLDFFDSYKIITCAESKFHNLVHDVDPMFYKMGEEYDVDTWRSPTHKQTTLELRYQYNDIYMLREVIEQFYIDGLCGGELPLTGMRTASSIAFNRLKKMTFGEEKTEEGYINYFELDKKTKFEFLRKRIEMESYTGGYTHANHKAVGKTINKIGCSLDINSAYPSQMAYKVFPYGKPVRKTWGRKPKTEKNEVYLIEVGFDFVEPKHEEYALDIFKIGAVNSKALSPITGAVSGQEYFCTNIKDGKAIPVYKELKDTKLTTNYNVVLTSVEYEFWIKHFNFGVFKKDEYDCFEVDNLEFTGLKIGSILYYKAEKGKFKFYVDHYMKMKVRNKKLGNRPLALQFKLILNGAYGKFGTGQNKEEKDLIMDKNGLLTFTGSVTKYEGKEFYRPYASFVTAYGRLQLWNAIIYAVGVENFLYCDTDSIYCNREVRSLIKDMNAIGETIDKTILGKWRVEHVFDKFKVLGQKKYMYHDCKQDYTDLKCCGLPYDARKIIIGQGFDEFYLGKNVEGKKQRKKVIGGCLLLDTLFTIKKIMF

SEQ ID NO: 40 - protein, 739 aa, synthetic construct
  REGION 1..739: note = 2582
MHHHHHHHHSGGSDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDKEEPKTFHIVQNNNIVYGCNVYMDKFFEVEWKDGSTTEIGLCLDFFDSYKIITCAESKFHNLVHDVDPMFYKMGEEYDVDTWRSPTHKQTTLELRYQYNDIYMLREVIEQFYIDGLCGGELPLTGMRTASSIAFNRLKKMTFGEEKTEEGYINYFELDKKTKFEFLRKRIEMESYTGGYTHANHKAVGKTINKIGCSLDINSAYPSQMAYKVFPYGKPVRKTWGRKPKTEKNEVYLIEVGFDFVEPKHEEYALDIFKIGAVNSKALSPITGAVSGQEYFCTNIKDGKAIPVYKELKDTKLTTNYNVVLTSVEYEFWIKHFNFGVFKKDEYDCFEVDNLEFTGLKIGSILYYKAEKGKFKFYVDHYMKMKVRNKKLGNRPLALQFKLILNGAYGKFGTGQNKEEKDLIMDKNGLLTFTGSVTKYEGKEFYRPYASFVTAYGRLQLWNAIIYAVGVENFLYCDTDSIYCNREVRSLIEDMNAIGETIDKTILGGWRVEHVFDKFKVLGQKKYMYHDCKQDYTDLKCCGLPYDARKIIIGQGFDEFYLGKNVEGKKQRKKVIGGCLLLDTLFTIKKIMF

SEQ ID NO: 41 - protein, 739 aa, synthetic construct
  REGION 1..739: note = 2583
MHHHHHHHHSGGSDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDKEEPKTFHIVQNNNIVYGCNVYMDKFFEVENKDGSTTEIGLCLDFFDSYKIITCAESKFHNLVHDVDPMFYKMGEEYDADTWRSPTHKQTTLELRYQYNDIYMLREVIEQFYIDGLCGGELPLTGMRTASSIAFLRLKKMTFGEEKTEEGYINYFELDKKTKFEFLRKRIEMESYHGGYTHANHKAVGKTINKIGCSLDINSAYPSQMAYKVFPYGKPVRKTWGRKPKTEKNEVYLIEVGFDFVEPKHEEYALDIFKIGAVNSKALSPITGAVSGQEYFCTNIKDGKAIPVYKELKDTKLTTNYNVVLTSVEYEFWIKHFNFGVFKKDEYDCFEVDNLEFTGLKIGSILYYKAEKGKFKFYVDHYMKMKVRNKKLGNRPLALQFKLILNGAYGKFGTGQNKEEKDLIMDKNGLLTFTGSVTKYEGKEFYRPYASFVTAYGRLQLWNAIIYAVGVENFLYCDTDSIYCNREVRSLIEDMNAIGETIDKTILGKWRVEHVFDKFKVLGQKKYMYHDCKQDYTDLKCCGLPYDARKIIIGQGFDEFYLGKNVEGKKQRKKVIGGCLLLDTLFTIKKIMF

SEQ ID NO: 42 - protein, 739 aa, synthetic construct
  REGION 1..739: note = 2584
MHHHHHHHHSGGSDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDKEEPKTFHIVQNNNIVYGCNVYMDKFFEVENKDGSTTEIGLCLDFFDSYKIITCAESKFHNLVHDVDPMFYKMGEEYDADTWRSPTHKQTTLELRYQYNDIYMLREVIEQFYIDGLCGGELPLTGMRTASSIAFNRLKKMTFGEEKTEEGYINYFELDKKTKFEFLRKRIEMESYHGGYTHANHKAVGKTINKIGCSLDINSAYPSQMAYKVFPYGKPVRKTWGRKPKTEKNEVYLIEVGFDFVEPKHEEYALDIFKIGAVNSKALSPITGAVSGQEYFCTNIKDGKAIPVYKELKDTKLTTNYNVVLTSVEYEFWIKHFNFGVFKKDEYDCFEVDNLEFTGLKIGSILYYKAEKGKFKFYVDHYMKMKVRNKKLGNRPLALQFKLILNGAYGKFGTGQNKEEKDLIMDKNGLLTFTGSVTKYEGKEFYRPYASFVTAYGRLQLWNAIIYAVGVENFLYCDTDSIYCNRFVRSLIEDMNAIGETIDKTILGKWRVEHVFDKFKVLGQKKYMYHDCKQDYTDLKCCGLPYDARKIIIGQGFDEFYLGKNVEGKKQRKKVIGGCLLLDTLFTIKKIMF

SEQ ID NO: 43 - protein, 739 aa, synthetic construct
  REGION 1..739: note = 2585
MHHHHHHHHSGGSDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDKEEPKTFHIVQNNNIVYGCNVYMDKFFEVENKDGSTTEIGLCLDFFDSYKIITCAESKFHNLVHDVDPMFYKMGEEYDADTWRSPTHKQTTLELRYQYNDIYMLREVIEQFYIDGLCGGELPLTGMRTASSIAFLRLKKMTFGEEKTEEGYINYFELDKKTKFEFLRKRIEMESYTGGYTHANHKAVGKTINKIGCSLDINSAYPSQMAYKVFPYGKPVRKTWGRKPKTEKNEVYLIEVGFDFVEPKHEEYALDIFKIGAVNSKALSPITGAVSGQEYFCTNIKDGKAIPVYKELKDTKLTTNYNVVLTSVEYEFWIKHFNFGVFKKDEYDCFEVDNLEFTGLKIGSILYYKAEKGKFKFYVIHYMKMKVRNKKLGNRPLALQFKLILNGAYGKFGTGQNKEEKDLIMDKNGLLTFTGSVTKYEGKEFYRPYASFVTAYGRLQLWNAIIYAVGVENFLYCDTDSIYCNREVRSLIEDMNAIGETIDKTILGKWRVEHVFDKFKVLGQKKYMYHDCKQDYTDLKCCGLPYDARKIIIGQGFDEFYLGKNVEGKKQRKKVIGGCLLLDTLFTIKKIMF

SEQ ID NO: 44 - protein, 739 aa, synthetic construct
  REGION 1..739: note = 2586
MHHHHHHHHSGGSDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDKEEPKTFHIVQNNNIVYGCNVYMDKFFEVENKDGSTTEIGLCLDFFDSYKIITCAESKFHNLVHDVDPMFYKMGEEYDADTWRSPTHKQTTLELRYQYNDIYMLREVIEQFYIDGLCGGELPLTGMRTASSIAFNRLKKMTFGEEKTEEGYINYFELDKKTKFEFLRKRIEMESYTGGYTHANHKAVGKTINKIGCSLDINSAYPSQMAYKVFPYGKPVRKTWGRKPKTEKNEVYLIEVGFDFVEPKHEEYALDIFKIGAVNSKALSPITGAVSGQEYFCTNIKDGKAIPVYKELKDTKLTTNYNVVLTSVEYEFWIKHFNFGVFKKDEYDCFEVDNLEFTGLKIGSILYYKAEKGKFKFYVIHYMKMKVRNKKRGNRPLALQFKLILNGAYGKFGTGQNKEEKDLIMDKNGLLTFTGSVTKYEGKEFYRPYASFVTAYGRLQLWNAIIYAVGVENFLYCDTDSIYCNREVRSLIEDMNAIGETIDKTILGKWRVEHVFDKFKVLGQKKYMYHDCKQDYTDLKCCGLPYDARKIIIGQGFDEFYLGKNVEGKKQRKKVIGGCLLLDTLFTIKKIMF

SEQ ID NO: 45 - protein, 739 aa, synthetic construct
  REGION 1..739: note = 2587
MHHHHHHHHSGGSDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDKEEPKTFHIVQNNNIVYGCNVYMDKFFEVENKDGSTTEIGLCLDFFDSYKIITCAESKFHNLVHDVDPMFYKMGEEYDADTWRSPTHKQTTLELRYQYNDIYMLREVIEQFYIDGLCGGELPLTGMRTASSIAFNRLKKMTFGEEKTEEGYINYFELDKKTKFEFLRKRIEMESYTGGYTHANHKAVGKTINKIGCSLDINSAYPSQMAYKVFPYGKPVRKTWGRKPKTEKNEVYLIEVGFDFVEPKHEEYALDIFKIGAVNSKALSPITGAVSGQEYFCTNIKDGKAIPVYKELKDTKLTTNYNVVLTSVEYEFWIKHFNFGVFKKDEYDCFEVDNLEFTGLKIGSILYYKAEKGKFKFYVIHYMKMKVRNKKLGNRPLALQFKLILNGAYGKFGTGQNKEEKDLHMDKNGLLTFTGSVTKYEGKEFYRPYASFVTAYGRLQLWNAIIYAVGVENFLYCDTDSIYCNREVRSLIEDMNAIGETIDKTILGKWRVEHVFDKFKVLGQKKYMYHDCKQDYTDLKCCGLPYDARKIIIGQGFDEFYLGKNVEGKKQRKKVIGGCLLLDTLFTIKKIMF

SEQ ID NO: 46 - protein, 739 aa, synthetic construct
  REGION 1..739: note = 2588
MHHHHHHHHSGGSDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDKEEPKTFHIVQNNNIVYGCNVYMDKFFEVENKDGSTTEIGLCLDFFDSYKIITCAESKFHNLVHDVDPMFYKMGEEYDADTWRSPTHKQTTLELRYQYNDIYMLREVIEQFYIDGLCGGELPLTGMRTASSIAFNRLKKMTFGEEKTEEGYINYFELDKKTKFEFLRKRIEMESYTGGYTHANHKAVGKTINKIGCSLDINSAYPSQMAYKVFPYGKPVRKTWGRKPKTEKNEVYLIEVGFDFVEPKHEEYALDIFKIGAVNSKALSPITGAVSGQEYFCTNIKDGKAIPVYKELKDTKLTTNYNVVLTSVEYEFWIKHFNFGVFKKDEYDCFEVDNLEFTGLKIGSILYYKAEKGKFKFYVIHYMKMKVRNKKLGNRPLALQFKLILNGAYGKFGTGQNKEEKDLGMDKNGLLTFTGSVTKYEGKEFYRPYASFVTAYGRLQLWNAIIYAVGVENFLYCDTDSIYCNREVRSLIEDMNAIGETIDKTILGKWRVEHVFDKFKVLGQKKYMYHDCKQDYTDLKCCGLPYDARKIIIGQGFDEFYLGKNVEGKKQRKKVIGGCLLLDTLFTIKKIMF

SEQ ID NO: 47 - protein, 739 aa, synthetic construct
  REGION 1..739: note = 2589
MHHHHHHHHSGGSDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDKEEPKTFHIVQNNNIVYGCNVYMDKFFEVENKDGSTTEIGLCLDFFDSYKIITCAESKFHNLVHDVDPMFYKMGEEYDADTWRSPTHKQTTLELRYQYNDIYMLREVIEQFYIDGLCGGELPLTGMRTASSIAFNRLKKMTFGEEKTEEGYINYFELDKKTKFEFLRKRIEMESYTGGYTHANHKAVGKTINKIGCSLDINSAYPSQMAYKVFPYGKPVRKTWGRKPKTEKNEVYLIEVGFDFVEPKHEEYALDIFKIGAVNSKALSPITGAVSGQEYFCTNIKDGKAIPVYKELKDTKLTTNYNVVLTSVEYEFWIKHFNFGVFKKDEYDCFEVDNLEFTGLKIGSILYYKAEKGKFKFYVIHYMKMKVRNKKLGNRPLALQFKLILNGAYGKFGTGQNKEEKDLIMDKNGLLTFTGSVTKYEGKEFYRPYASFVTAYGRLQLWNAIIYAVGVENFLYCDTDSIYCNRFVRSLIEDMNAIGETIDKTILGKWRVEHVFDKFKVLGQKKYMYHDCKQDYTDLKCCGLPYDARKIIIGQGFDEFYLGKNVEGKKQRKKVIGGCLLLDTLFTIKKIMF

SEQ ID NO: 48 - protein, 739 aa, synthetic construct
  REGION 1..739: note = 2590
MHHHHHHHHSGGSDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDKEEPKTFHIVQNNNIVYGCNVYMDKFFEVENKDGSTTEIGLCLDFFDSYKIITCAESKFHNLVHDVDPMFYKMGEEYDADTWRSPTHKQTTLELRYQYNDIYMLREVIEQFYIDGLCGGELPLTGMRTASSIAFNRLKKMTFGEEKTEEGYINYFELDKKTKFEFLRKRIEMESYTGGYTHANHKAVGKTINKIGCSLDINSAYPSQMAYKVFPYGKPVRKTWGRKPKTEKNEVYLIEVGFDFVEPKHEEYALDIFKIGAVNSKALSPITGAVSGQEYFCTNIKDGKAIPVYKELKDTKLTTNYNVVLTSVEYEFWIKHFNFGVFKKDEYDCFEVDNLEFTGLKIGSILYYKAEKGKFKFYVIHYMKMKVRNKKLGNRPLALQFKLILNGAYGKFGTGQNKEEKDLIMDKNGLLTFTGSVTKYEGKEFYRPYASFVTAYGRLQLWNAIIYAVGVENFLYCDTDSIYCNREVRSLIKDMNAIGETIDKTILGKWRVEHVFDKFKVLGQKKYMYHDCKQDYTDLKCCGLPYDARKIIIGQGFDEFYLGKNVEGKKQRKKVIGGCLLLDTLFTIKKIMF

SEQ ID NO: 49 - protein, 739 aa, synthetic construct
  REGION 1..739: note = 2591
MHHHHHHHHSGGSDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDKEEPKTFHIVQNNNIVYGCNVYMDKFFEVENKDGSTTEIGLCLDFFDSYKIITCAESKFHNLVHDVDPMFYKMGEEYDADTWRSPTHKQTTLELRYQYNDIYMLREVIEQFYIDGLCGGELPLTGMRTASSIAFNRLKKMTFGEEKTEEGYINYFELDKKTKFEFLRKRIEMESYTGGYTHANHKAVGKTINKIGCSLDINSAYPSQMAYKVFPYGKPVRKTWGRKPKTEKNEVYLIEVGFDFVEPKHEEYALDIFKIGAVNSKALSPITGAVSGQEYFCTNIKDGKAIPVYKELKDTKLTTNYNVVLTSVEYEFWIKHFNFGVFKKDEYDCFEVDNLEFTGLKIGSILYYKAEKGKFKFYVIHYMKMKVRNKKLGNRPLALQFKLILNGAYGKFGTGQNKEEKDLIMDKNGLLTFTGSVTKYEGKEFYRPYASFVTAYGRLQLWNAIIYAVGVENFLYCDTDSIYCNREVRSLIEDMNAIGETIDKTILGGWRVEHVFDKFKVLGQKKYMYHDCKQDYTDLKCCGLPYDARKIIIGQGFDEFYLGKNVEGKKQRKKVIGGCLLLDTLFTIKKIMF

SEQ ID NO: 50 - protein, 293 aa, synthetic construct
  REGION 1..293: note = Variant aHL monomer G1742
ADSDINIKTGTTDIGSNTTVKTGDLVTYDKENGMGKKVFYSFIDDKKHNKKLLVIRTKGTIAGQYRVYSEEGANKSGLAWPSAFKVQLQLPDNEVAQISDYYPRNSIDTKNYASTLTYGFNGNVTGGKGGGIGGLIGANVSIGATLNYKQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 51 - protein, 293 aa, synthetic construct
  REGION 1..293: note = Variant aHL monomer G1678
ADSDINIKTGTTDIGSNTTVKTGDLVTYDKENGMGKKVFYSFIDDKNHNKKLLVIRTKGTIAGQYRVYSEEGANKSGLAWPSAFKVQLQLPDNEVAQISDYYPRNSIDTKNYASTLTYGFNGNVTGGGGGKIGGLIGANVSIGHTLNYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

(19) United States
(12) Patent Application Publication
(10) Pub. No.: US 2025/0019676 A1
(43) Pub. Date: Jan. 16, 2025

(54) THERMOSTABLE POLYMERASE VARIANTS

(71) Applicant: Roche Sequencing Solutions, Inc., Pleasanton, CA (US)

(72) Inventors: Cleoma Arnold, San Jose, CA (US); Aruna Ayer, Santa Clara, CA (US); David Beyer, Oakland, CA (US); Neha Jain, Fremont, CA (US); Naga Kishore Kakani, Fremont, CA (US); Yarrow Madrona, Pleasanton, CA (US); Ashtamurthy Pawate, San Jose, CA (US); David Penkler, San Ramon, CA (US); Christopher Witbooi, Cape Town (ZA)

(21) Appl. No.: 18/712,051

(22) PCT Filed: Nov. 21, 2022
(86) PCT No.: PCT/EP2022/082628

§ 371 Date: May 21, 2024

Related U.S. Application Data
(60) Provisional application No. 63/281,861, filed on Nov. 22, 2021.

(51) Int. Cl. C12N 9/12; G01N 27/327

(52) U.S. Cl. CPC C12N 9/1252; G01N 27/3278; C12Y 207/07007

(57) ABSTRACT

Described herein is a variant pol6 polymerase having improved thermostability. The disclosed polymerases each contain one or more substitutions relative to SEQ ID NO: 1 selected from the group consisting of G12W/Y/F, K114W/F/I, L117L/F/P, N194F/W/V, M232W/G/R, G313E/Q/L, A451F/W/Y, K490W/F/Y, Q565Y/I/V, Q590P/V/Y, and D681G/N/H. Additionally, the present specification discloses substitutions that can reduce stuttering in such polymerases, the substitutions selected from the group consisting of N298L, L538R, P542A, I570H/T/W/R/N/G, N574L, E633W/F, S636F, E639K, and K655G.

### TECHNICAL FIELD

Provided herein, among other things, are modified DNA polymerases containing amino acid alterations based on mutations identified in directed evolution experiments designed to select enzymes that are better suited for applications in recombinant DNA technologies.

### BACKGROUND

DNA polymerases are a family of enzymes that use single-stranded DNA as a template to synthesize the complementary DNA strand. In particular, DNA polymerases can add free nucleotides to the 3′ end of a newly-forming strand resulting in elongation of the new strand in a 5′ to 3′ direction. Most DNA polymerases are multifunctional proteins that possess both polymerizing and exonucleolytic activities. For example, many DNA polymerases have 3′→5′ exonuclease activity. These polymerases can recognize an incorrectly incorporated nucleotide and the 3′→5′ exonuclease activity of the enzyme allows the incorrect nucleotide to be excised (this activity is known as proofreading). Following nucleotide excision, the polymerase can re-insert the correct nucleotide and replication can continue. Many DNA polymerases also have 5′→3′ exonuclease activity.

Polymerases have found use in recombinant DNA applications, including nanopore sequencing. However, a DNA strand moves rapidly at the rate of 1 s to 5 s per base through the nanopore. This makes recording difficult and prone to background noise, failing in obtaining single-nucleotide resolution. Therefore, the use of detectable tags on nucleotides may be used in the sequencing of a DNA strand or fragment thereof. Thus, there is not only a need to control the rate of DNA being sequenced but also provide polymerases that have improved properties (relative to the wild-type enzyme) such as incorporation of modified nucleotides, e.g., polyphosphate nucleotides with or without tags.

### BRIEF SUMMARY OF THE DISCLOSURE

The present disclosure provides modified DNA polymerases (e.g., mutants) based on directed evolution experiments designed to select mutations that confer advantageous phenotypes under conditions used in industrial or research applications. In particular, two sets of sites for substituting Pol6 polymerases to obtain beneficial effects have been identified.

Substitution at the first set of sites can confer improved thermostability, including substitution sites G12, K114, L117, N194, M232, N298, G313, A451, K490, L538, P542, Q565, 1570, N574L, Q590, E633, S636, E639, K655, and D681 relative to SEQ ID NO: 2. Specific substitutions that can be used at these sites include G12 W/Y/F, K114 W/F/I, L117L/F/P, N194F/W/V, M232 W/G/R, G313E/Q/L, A451F/W/Y, K490 W/F/Y, Q565Y/I/V, Q590P/V/Y, and D681G/N/H substitutions relative to SEQ ID NO: 2.

In a specific embodiment, the isolated polypeptide includes at least one substitution selected from the group consisting of G12 W, G12Y, G12F, A451F, A451 W, N194 W, E5651, E590Y, and D681H.

Another embodiment of the disclosure is an isolated polypeptide as described herein, wherein the at least one substitution is selected from the group consisting of A451F, N194 W, D681H.

Other objects, features and advantages of the present disclosure will become apparent from the following detailed description. It should be understood, however, that the detailed description and specific examples, while indicating preferred embodiments of the disclosure, are given by way of illustration only, since various changes and modifications within the scope and spirit of the disclosure will become apparent to one skilled in the art from this detailed description.

### BRIEF DESCRIPTION OF THE DRAWINGS

The file of this patent contains at least one drawing in color. Copies of this patent or patent publication with color drawing(s) will be provided by the Office upon request and payment of the necessary fee.

FIG. 1A illustrates the active fraction of numerous polymerases at between 39° C. and 40° C. (left graph) and at 46° C.

FIG. 1B illustrates the active fraction of numerous polymerases at between 30° C. and 46° C. (a) highlights polymerases with higher stability than the parental polymerase at around 30° C. (b) highlights polymerases with higher stability than the parental polymerase at around 38° C. (c) highlights polymerases with higher stability than the parental polymerase at around 46° C.

FIG. 1C summarizes the breakdown of active polymerases based on improved thermostability compared to the parental polymerase.

FIG. 2 summarizes the on-chip thermostability of downselected polymerases from Example 1.

FIG. 3A is a heat map comparing variant kinetics relative to Pol6-1743 for each of the following characteristics: (A) average waiting time, (B) mean dwell time, (C) mean stuttering, (D) mean stuttering rate, (E) percentage of insertions, (F) percentage of deletions, (G) accuracy, (H) procession rate, (I) procession length, and (J) sequencing lifetime.

FIG. 3B illustrates the on-chip active fraction of each variant evaluated in FIG. 3A as compared to Pol6-1743 at various temperatures between 3° and 46° C.

FIG. 4 illustrates on-chip kinetics of Pol6-1743 (A), Pol6-2269 (B), and Pol6-2271 (C) at each of 36, 38, and 40° C. The top graph illustrates the on-chip active fraction and the bottom graph illustrates the median read length.

FIG. 5A illustrates on-chip length profile of 5 variants (Pol6-2565, Pol6-2570, Pol6-2571, Pol6-2573 and Pol6-2579) with either plasmid or E. Coli genomic templates.

FIG. 5B on-chip accuracy of 5 variants (Pol6-2565, Pol6-2570, Pol6-2571, Pol6-2573 and Pol6-2579) with either plasmid or E. Co/i genomic templates.

FIG. 5C illustrates on-chip deletions (left graph) and insertion profiles of 5 variants (Pol6-2565, Pol6-2570, Pol6-2571, Pol6-2573 and Pol6-2579) with either plasmid or E. Coli genomic templates.

### DETAILED DESCRIPTION

The disclosure will now be described in detail by way of reference only using the following definitions and examples. All patents and publications, including all sequences disclosed within such patents and publications, referred to herein are expressly incorporated by reference.

Unless defined otherwise herein, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 2D ED., John Wiley and Sons, New York (1994), and Hale & Marham, THE HARPER COLLINS DICTIONARY OF BIOLOGY, Harper Perennial, NY (1991) provide one of skill with a general dictionary of many of the terms used in this disclosure. Although any methods and materials similar or equivalent to those described herein can be used in the practice or testing of the present disclosure, specific methods and materials are described. Practitioners are particularly directed to Sambrook et al., 1989, and Ausubel F M et al., 1993, for definitions and terms of the art. It is to be understood that this disclosure is not limited to the particular methodology, protocols, and reagents described, as these may vary.

Numeric ranges are inclusive of the numbers defining the range. The term “about” is used herein to mean plus or minus ten percent (10%) of a value. For example, “about 100” refers to any number between 90 and 110.

Unless otherwise indicated, nucleic acids are written left to right in 5′ to 3′ orientation; amino acid sequences are written left to right in amino to carboxy orientation, respectively.

The headings provided herein are not limitations of the various aspects or embodiments of the disclosure which can be had by reference to the specification as a whole. Accordingly, the terms defined immediately below are more fully defined by reference to the specification as a whole.

### Definitions

Amino acid: As used herein, the term “amino acid,” in its broadest sense, refers to any compound and/or substance that can be incorporated into a polypeptide chain. In some embodiments, an amino acid has the general structure H₂N C(H)(R)—COOH. In some embodiments, an amino acid is a naturally-occurring amino acid. In some embodiments, an amino acid is a synthetic amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, and/or substitution with other chemical without adversely affecting their activity. Amino acids may participate in a disulfide bond. The term “amino acid” is used interchangeably with “amino acid residue,” and may refer to a free amino acid and/or to an amino acid residue of a peptide. It will be apparent from the context in which the term is used whether it refers to a free amino acid or a residue of a peptide. It should be noted that all amino acid residue sequences are represented herein by formulae whose left and right orientation is in the conventional direction of amino-terminus to carboxy-terminus.

Base Pair (bp): As used herein, base pair refers to a partnership of adenine (A) with thymine (T), or of cytosine (C) with guanine (G) in a double stranded DNA molecule.

Complementary: As used herein, the term “complementary” refers to the broad concept of sequence complementarity between regions of two polynucleotide strands or between two nucleotides through base-pairing. It is known that an adenine nucleotide is capable of forming specific hydrogen bonds (“base pairing”) with a nucleotide which is thymine or uracil. Similarly, it is known that a cytosine nucleotide is capable of base pairing with a guanine nucleotide.

DNA binding affinity: As used herein, the term “DNA-binding affinity” typically refers to the activity of a DNA polymerase in binding DNA nucleic acid. In some embodiments, DNA binding activity can be measured in a two band-shift assay. See, e.g., Sambrook et al. (2001) Molecular Cloning: A Laboratory Manual (3^{rd} ed., Cold Spring Harbor Laboratory Press, NY) at 9.63-9.75 (describing end-labeling of nucleic acids). A reaction mixture is prepared containing at least about 0.5 μg of the polypeptide in about 10 μl of binding buffer (50 mM sodium phosphate buffer (pH 8.0), 10% glycerol, 25 mM KCl, 25 mM MgCl₂). The reaction mixture is heated to 37° C. for 10 min. About 1×10⁴ to 5×10⁴ cpm (or about 0.5-2 ng) of the labeled double-stranded nucleic acid is added to the reaction mixture and incubated for an additional 10 min. The reaction mixture is loaded onto a native polyacrylamide gel in 0.5× Tris-borate buffer. The reaction mixture is subjected to electrophoresis at room temperature. The gel is dried and subjected to autoradiography using standard methods. Any detectable decrease in the mobility of the labeled double-stranded nucleic acid indicates formation of a binding complex between the polypeptide and the double-stranded nucleic acid. Such nucleic acid binding activity may be quantified using standard densitometric methods to measure the amount of radioactivity in the binding complex relative to the total amount of radioactivity in the initial reaction mixture. Other methods of measuring DNA binding affinity are known in the art (see, e.g., Kong et al. (1993) J. Biol. Chem. 268(3):1965-1975).

DNA polymerase activity: As used herein, the term “DNA polymerase activity” refers to the ability to catalyze the polymerization of deoxyribonucleotides into a DNA molecule in a template-dependent manner. DNA polymerase activity can be measured using various techniques and methods known in the art. For example, serial dilutions of polymerase can be prepared in dilution buffer (e.g., 20 mM Tris HCl, pH 8.0, 50 mM KCl, 0.5% NP 40, and 0.5% Tween-20). For each dilution, 5 μl can be removed and added to 45 μl of a reaction mixture containing 25 mM TAPS (pH 9.25), 50 mM KCl, 2 mM MgCl₂, 0.2 mM dATP, 0.2 mM dGTP, 0.2 mM dTTP, 0.1 mM dCTP, 12.5 μg activated DNA, 100 μM (α-³²P)dCTP (0.05 μCi/nmol) and sterile deionized water. The reaction mixtures can be incubated at 37° C. (or 74° C. for thermostable DNA polymerases) for 10 minutes and then stopped by immediately cooling the reaction to 4° C. and adding 10 μl of ice-cold 60 mM EDTA. A 25 μl aliquot can be removed from each reaction mixture. Unincorporated radioactively labeled dCTP can be removed from each aliquot by gel filtration (Centri-Sep, Princeton Separations, Adelphia, N.J.). The column eluate can be mixed with scintillation fluid (1 ml). Radioactivity in the column eluate is quantified with a scintillation counter to determine the amount of product synthesized by the polymerase. One unit of polymerase activity can be defined as the amount of polymerase necessary to synthesize 10 nmole of product in 30 minutes (Lawyer et al. (1989) J. Biol. Chem. 264:6427-647). Other methods of measuring polymerase activity are known in the art (see, e.g. Sambrook et al. (2001) Molecular Cloning: A Laboratory Manual (3rd ed., Cold Spring Harbor Laboratory Press, NY)).

Procession length: As used herein, the term “procession length” refers to the average length of polymer chain that can be generated by DNA polymerase. Sometimes also referred to as “elongation length”, “extension length” and “incorporation length.”

Procession rate: As used herein, the term “procession rate” refers to the average rate at which a DNA polymerase extends a polymer chain. Sometimes also referred to as “elongation rate”, “extension rate” and “incorporation rate.”

Purified: As used herein, “purified” means that a molecule is present in a sample at a concentration of at least 90% by weight, or at least 95% by weight, or at least 98% by weight of the sample in which it is contained.

Isolated: An “isolated” molecule is a nucleic acid molecule that is separated from at least one other molecule with which it is ordinarily associated, for example, in its natural environment. An isolated nucleic acid molecule includes a nucleic acid molecule contained in cells that ordinarily express the nucleic acid molecule, but the nucleic acid molecule is present extrachromasomally or at a chromosomal location that is different from its natural chromosomal location.

Percent (%) homology: The term “% homology” is used interchangeably herein with the term “% identity” herein and refers to the level of nucleic acid or amino acid sequence identity between the nucleic acid sequence that encodes any one of the inventive polypeptides or the inventive polypeptide's amino acid sequence, when aligned using a sequence alignment program.

For example, as used herein, 80% homology means the same thing as 80% sequence identity determined by a defined algorithm, and accordingly a homologue of a given sequence has greater than 80% sequence identity over a length of the given sequence. Exemplary levels of sequence identity include, but are not limited to, 80, 85, 90, 95, 98% or more sequence identity to a given sequence, e.g., the coding sequence for any one of the inventive polypeptides, as described herein.

Exemplary computer programs which can be used to determine identity between two sequences include, but are not limited to, the suite of BLAST programs, e.g., BLASTN, BLASTX, and TBLASTX, BLASTP and TBLASTN, publicly available on the Internet. See also, Altschul, et al., 1990 and Altschul, et al., 1997.

Sequence searches are typically carried out using the BLASTN program when evaluating a given nucleic acid sequence relative to nucleic acid sequences in the GenBank DNA Sequences and other public databases. The BLASTX program can be used for searching nucleic acid sequences that have been translated in all reading frames against amino acid sequences in the GenBank Protein Sequences and other public databases. Both BLASTN and BLASTX are run using default parameters of an open gap penalty of 11.0, and an extended gap penalty of 1.0, and utilize the BLOSUM-62 matrix. (See, e.g., Altschul, S. F., et al., Nucleic Acids Res. 25:3389-3402, 1997.)

An alignment of selected sequences in order to determine “% identity” between two or more sequences, is performed using for example, the CLUSTAL-W program in MacVector version 13.0.7, operated with default parameters, including an open gap penalty of 10.0, an extended gap penalty of 0.1, and a BLOSUM 30 similarity matrix.

Modified DNA polymerase: As used herein, the term “modified DNA polymerase” refers to a DNA polymerase originated from another (i.e., parental) DNA polymerase and contains one or more amino acid alterations (e.g., amino acid substitution, deletion, or insertion) compared to the parental DNA polymerase. In some embodiments, a modified DNA polymerases of the disclosure is originated or modified from a naturally-occurring or wild-type DNA polymerase. In some embodiments, a modified DNA polymerase of the disclosure is originated or modified from a recombinant or engineered DNA polymerase including, but not limited to, chimeric DNA polymerase, fusion DNA polymerase or another modified DNA polymerase. Typically, a modified DNA polymerase has at least one changed phenotype compared to the parental polymerase.

Mutation: As used herein, the term “mutation” refers to a change introduced into a parental sequence, including, but not limited to, substitutions, insertions, deletions (including truncations). The consequences of a mutation include, but are not limited to, the creation of a new character, property, function, phenotype or trait not found in the protein encoded by the parental sequence.

Mutant: As used herein, the term “mutant” refers to a modified protein which displays altered characteristics when compared to the parental protein. The terms “variant” and “mutant” are used interchangeably herein.

Wild-type: As used herein, the term “wild-type” refers to a gene or gene product which has the characteristics of that gene or gene product when isolated from a naturally-occurring source.

Fidelity: As used herein, the term “fidelity” refers to either the accuracy of DNA polymerization by template-dependent DNA polymerase or the measured difference in k_{off} of the correct nucleotide vs incorrect nucleotide binding to the template DNA. The fidelity of a DNA polymerase is typically measured by the error rate (the frequency of incorporating an inaccurate nucleotide, i.e., a nucleotide that is not incorporated at a template-dependent manner). The accuracy or fidelity of DNA polymerization is maintained by both the polymerase activity and the 3′-5′ exonuclease activity of a DNA polymerase. The term “high fidelity” refers to an error rate less than 4.45×10⁻⁶ (e.g., less than 4.0×10⁻⁶, 3.5×10⁻⁶, 3.0×10⁻⁶, 2.5×10⁻⁶, 2.0×10⁻⁶, 1.5×10⁻⁶, 1.0×10⁻⁶, 0.5×10⁻⁶) mutations/nt/doubling. The fidelity or error rate of a DNA polymerase may be measured using assays known to the art. For example, the error rates of DNA polymerases can be tested as described herein or as described in Johnson, et al., Biochim Biophys Acta. 2010 May; 1804(5): 1041-1048.

Nanopore: The term “nanopore,” as used herein, generally refers to a pore, channel or passage formed or otherwise provided in a membrane. A membrane may be an organic membrane, such as a lipid bilayer, or a synthetic membrane, such as a membrane formed of a polymeric material. The membrane may be a polymeric material. The nanopore may be disposed adjacent or in proximity to a sensing circuit or an electrode coupled to a sensing circuit, such as, for example, a complementary metal-oxide semiconductor (CMOS) or field effect transistor (FET) circuit. In some examples, a nanopore has a characteristic width or diameter on the order of 0.1 nanometers (nm) to about 1000 nm. Some nanopores are proteins. Alpha-hemolysin, MspA are examples of a protein nanopore.

Nucleotide: As used herein, a monomeric unit of DNA or RNA consisting of a sugar moiety (pentose), a phosphate, and a nitrogenous heterocyclic base. The base is linked to the sugar moiety via the glycosidic carbon (1′ carbon of the pentose) and that combination of base and sugar is a nucleoside. When the nucleoside contains a phosphate group bonded to the 3′ or 5′ position of the pentose it is referred to as a nucleotide. A sequence of operatively linked nucleotides is typically referred to herein as a “base sequence” or “nucleotide sequence,” and is represented herein by a formula whose left to right orientation is in the conventional direction of 5′-terminus to 3′-terminus. As used herein, a “modified nucleotide” refers to a polyphosphate, e.g., 3, 4, 5, 6, 7 or 8 phosphates, nucleotide.

Oligonucleotide or Polynucleotide: As used herein, the term “oligonucleotide” is defined as a molecule including two or more deoxyribonucleotides and/or ribonucleotides, e.g., more than three. Its exact size will depend on many factors, which in turn depend on the ultimate function or use of the oligonucleotide. The oligonucleotide may be derived synthetically or by cloning. As used herein, the term “polynucleotide” refers to a polymer molecule composed of nucleotide monomers covalently bonded in a chain. DNA (deoxyribonucleic acid) and RNA (ribonucleic acid) are examples of polynucleotides.

Polymerase: As used herein, a “polymerase” refers to an enzyme that catalyzes the polymerization of nucleotide (i.e., the polymerase activity). Generally, the enzyme will initiate synthesis at the 3′-end of the primer annealed to a polynucleotide template sequence, and will proceed toward the 5′ end of the template strand. A “DNA polymerase” catalyzes the polymerization of deoxynucleotides.

Primer: As used herein, the term “primer” refers to an oligonucleotide, whether occurring naturally or produced synthetically, which is capable of acting as a point of initiation of nucleic acid synthesis when placed under conditions in which synthesis of a primer extension product which is complementary to a nucleic acid strand is induced, e.g., in the presence of four different nucleotide triphosphates and thermostable enzyme in an appropriate buffer (“buffer” includes pH, ionic strength, cofactors, etc.) and at a suitable temperature. In one embodiment, the primer is single-stranded for maximum efficiency in amplification, but may alternatively be double-stranded. If double-stranded, the primer is first treated to separate its strands before being used to prepare extension products. In a specific embodiment, the primer is an oligodeoxyribonucleotide. The primer must be sufficiently long to prime the synthesis of extension products in the presence of the thermostable enzyme. The exact lengths of the primers will depend on many factors, including temperature, source of primer and use of the method. For example, depending on the complexity of the target sequence, the oligonucleotide primer typically contains 15-25 nucleotides, although it may contain more or few nucleotides. Short primer molecules generally require colder temperatures to form sufficiently stable hybrid complexes with template.

Processivity: As used herein, “processivity” refers to the ability of a polymerase to remain attached to the template and perform multiple modification reactions. “Modification reactions” include but are not limited to polymerization, and exonucleolytic cleavage. In some embodiments, “processivity” refers to the ability of a DNA polymerase to perform a sequence of polymerization steps without intervening dissociation of the enzyme from the growing DNA chains. Typically, “processivity” of a DNA polymerase is measured by the length of nucleotides (for example 20 nts, 300 nts, 0.5-1 kb, or more) that are polymerized or modified without intervening dissociation of the DNA polymerase from the growing DNA chain. “Processivity” can depend on the nature of the polymerase, the sequence of a DNA template, and reaction conditions, for example, salt concentration, temperature or the presence of specific proteins. As used herein, the term “high processivity” refers to a processivity higher than 20 nts (e.g., higher than 40 nts, 60 nts, 80 nts, 100 nts, 120 nts, 140 nts, 160 nts, 180 nts, 200 nts, 220 nts, 240 nts, 260 nts, 280 nts, 300 nts, 320 nts, 340 nts, 360 nts, 380 nts, 400 nts, or higher) per association/disassociation with the template. Processivity can be measured according the methods defined herein and in WO 01/92501 A1 (MJ Bioworks, Inc., Improved Nucleic Acid Modifying Enzymes, published 6 Dec. 2001).

Stuttering: As used herein, the term “stuttering” refers to insertions in an amplicon relative to the template from which the amplicon is derived during a DNA-polymerase catalyzed sequencing reaction. When used as a kinetic measurement of a DNA-polymerase, “stuttering” shall refer to the absolute number of insertions generated in a given sequencing run, while the “stuttering rate” shall refer to the fraction of nucleotides polymerized that are insertions relative to the template nucleic acid.

Synthesis: As used herein, the term “synthesis” refers to any in vitro method for making new strand of polynucleotide or elongating existing polynucleotide (i.e., DNA or RNA) in a template dependent manner Synthesis, according to the disclosure, includes amplification, which increases the number of copies of a polynucleotide template sequence with the use of a polymerase. Polynucleotide synthesis (e.g., amplification) results in the incorporation of nucleotides into a polynucleotide (i.e., a primer), thereby forming a new polynucleotide molecule complementary to the polynucleotide template. The formed polynucleotide molecule and its template can be used as templates to synthesize additional polynucleotide molecules. “DNA synthesis,” as used herein, includes, but is not limited to, PCR, the labeling of polynucleotide (i.e., for probes and oligonucleotide primers), polynucleotide sequencing.

Template DNA molecule: As used herein, the term “template DNA molecule” refers to a strand of a nucleic acid from which a complementary nucleic acid strand is synthesized by a DNA polymerase, for example, in a primer extension reaction.

Template-dependent manner: As used herein, the term “template-dependent manner” refers to a process that involves the template dependent extension of a primer molecule (e.g., DNA synthesis by DNA polymerase). The term “template-dependent manner” typically refers to polynucleotide synthesis of RNA or DNA wherein the sequence of the newly synthesized strand of polynucleotide is dictated by the well-known rules of complementary base pairing (see, for example, Watson, J. D. et al., In: Molecular Biology of the Gene, 4th Ed., W. A. Benjamin, Inc., Menlo Park, Calif. (1987)).

Tag: As used herein, the term “tag” refers to a detectable moiety that may be atoms or molecules, or a collection of atoms or molecules. A tag may provide an optical, electrochemical, magnetic, or electrostatic (e.g., inductive, capacitive) signature, which signature may be detected with the aid of a nanopore.

Tagged Nucleotide: As used herein, the term “tagged nucleotide” refers to a nucleotide or modified nucleotide that has a tag attached. The tag may be attached covalently to the sugar, the phosphate (or polyphosphate) or base. The tag may be on the terminal phosphate.

Vector: As used herein, the term “vector” refers to a nucleic acid construct designed for transfer between different host cells. An “expression vector” refers to a vector that has the ability to incorporate and express heterologous DNA fragments in a foreign cell. Many prokaryotic and eukaryotic expression vectors are commercially available. Selection of appropriate expression vectors is within the knowledge of those having skill in the art.

The polymerase variants provided for herein are useful in the chip-based polynucleotide sequencing as described in WO2013/188841, US 2018-0306746A1, U.S. Pat. Nos. 10,174,371, and 10,036,739, among many others.

### Nomenclature

In the present description and claims, the conventional one-letter and three-letter codes for amino acid residues are used.

For ease of reference, polymerase variants of the application are described by use of the following nomenclature:

Original amino acid(s): position(s): substituted amino acid(s). According to this nomenclature, for instance the substitution of serine by an alanine in position 242 is shown as:
Ser242Ala or S242A

Multiple mutations are separated by plus signs, i.e.:
Ala30Asp+Glu34Ser or A30N+E34S
representing mutations in positions 30 and 34 substituting alanine and glutamic acid for asparagine and serine, respectively.

When one or more alternative amino acid residues may be inserted in a given position it is indicated as: A30N/E or A30N or A30E.

Unless otherwise stated, the number of the residues corresponds to the residue numbering of SEQ ID NO:2.

### Variant Pol6 Polymerases

Isolated polypeptides having DNA polymerase activity that are derived from Pol6 of Clostridium phage phiCPV4 (referred to hereafter as “Pol6”) are provided. A wild type amino acid sequence for Pol6 is disclosed herein as SEQ ID NO: 1 and a His-tagged version of the same is disclosed at SEQ ID NO: 2. The isolated polypeptides have DNA polymerase activity and a substitution at one or more positions of SEQ ID NO: 2 selected from the group consisting of G12, K114, L117, N194, M232, N298, G313, A451, K490, L538, P542, Q565, 1570, N574L, Q590, E633, S636, E639, K655, and D681. The isolated polypeptides can be generated by site-directed mutagenesis of a nucleic acid encoding a parental polypeptide. Methods of site-directed mutagenesis are well-known in the art and any method that reliably generates the desired mutated polypeptide may be used. Any polypeptide derived from Pol6 may be used as the parental polypeptide. Numerous such polypeptides are known in the art, including those disclosed in US 2016-0222363 A1, US 2016-0333327 A1, US 2018-0245147 A1, US 2017-0267983 A1, and US 2018-0094249 (each of which incorporated herein by reference in its entirety). Exemplary parental polypeptides include WT-Pol6 (SEQ ID NO: 1), His-Tagged Pol6 (SEQ ID NO: 2), Pol6-P1743 (SEQ ID NO: 3), Pol6-2094 (SEQ ID NO: 4), Pol6-2271 (SEQ ID NO: 5), Pol6-2303 (SEQ ID NO: 6), Pol6-2546 (SEQ ID NO: 7), Pol6-2569 (SEQ ID NO: 8), Pol6-2570 (SEQ ID NO: 9), Pol6-2571 (SEQ ID NO: 10), Pol6-2573 (SEQ ID NO: 11), and Pol6-2579 (SEQ ID NO: 12).

In an embodiment, the isolated polypeptide comprises an amino acid sequence that has at least 75%, at least 80%, at least 85%, at least 90%, or at least 95% sequence identity to amino acid residues 11-273 of SEQ ID NO: 2, with the proviso that the amino acid sequence has a substitution at one or more positions relative to SEQ ID NO: 2 selected from the group consisting of G12, K114, L117, N194, M232, N298, G313, A451, K490, L538, P542, Q565, 1570, N574L, Q590, E633, S636, E639, K655, and D681. It should be noted that the sequence identity is based upon on amino acid residues 11-273 of SEQ ID NO: 2. Amino acid residues 1-10 are a part of a His-tag and do not materially affect the function of the DNA polymerase. Moreover, other entities may be inserted between residues 10 and 11 (among other locations), such as additional affinity tags, labels, or entities useful in purification (such as a SpyCatcher peptide or a SpyTag peptide). An example of this is a SpyCatcher-tagged version of Pol6-P2271 (SEQ ID NO: 13), which corresponds to SEQ ID NO: 5 that has a SpyCatcher polypeptide (residues 11-112 of SEQ ID NO: 13) inserted between residues 10 and 11.

Positions G12, K114, L117, N194, M232, G313, A451, K490, Q565, Q590, and D681 were identified as candidates for modification by intra-protein residue interaction mapping as sites that could be modified to help improve the thermostability of the polypeptide's DNA polymerase activity. Substitutions at these sites may be chosen such that the polypeptide has an improved thermostability at 36° C. or greater relative to a reference polypeptide. Unless otherwise stated, the reference polypeptide is Pol6-1743 (SEQ ID NO: 3). As used herein, and “increased thermostability relative to a reference polypeptide at a temperature in the range of 37° C. to 47° C.” shall mean that the active fraction of the subject polypeptide is higher than the active fraction of the reference polypeptide at least at one temperature in the range of 37° C. to 47° C. Thus, for example, if the subject polypeptide has a higher active fraction than the reference polypeptide at 37° C., but not at 47° C., it will still be considered to have “increased thermostability.” For the purposes of determining whether a polypeptide has the described improvement in thermostability, the active fraction is determined as described in Example 2. In an embodiment, the substitution(s) is/are selected such that the polypeptide has at least 1.5-fold greater thermostability of DNA polymerase activity relative to Pol6-1743 at 36° C., 38° C., 40° C., and/or 46° C. In another embodiment, the substitution(s) is/are selected such that the polypeptide has at least 2-fold greater thermostability of DNA polymerase activity relative to Pol6-1743 at 36° C. In another embodiment, the substitution(s) is/are selected such that the polypeptide has at least 2-fold greater thermostability of DNA polymerase activity relative to Pol6-1743 at 38° C. In another embodiment, the substitution(s) is/are selected such that the polypeptide has at least 3-fold greater thermostability of DNA polymerase activity relative to Pol6-1743 at 38° C. In another embodiment, the substitution(s) is/are selected such that the polypeptide has a greater thermostability of DNA polymerase activity than Pol6-1743 at 46° C. In another embodiment, the substitution(s) is/are selected such that the polypeptide has at least 1.5-fold greater thermostability of DNA polymerase activity relative to Pol6-1743 at 46° C.

Exemplary substitutions at these sites include G12 W/Y/F, K114 W/F/I, L117L/F/P, N194F/W/V, M232 W/G/R, G313E/Q/L, A451F/W/Y, K490 W/F/Y, Q565Y/I/V, Q590P/V/Y, and D681G/N/H.

Positions N298, L538, P542, 1570, N574, E633, S636, E639, and K655 were identified as candidates for modification by mining a database of historical Pol6 substitutions to identify positions that could be modified to obtain desired stuttering rates and/or procession lengths. For the purposes of determining whether a polypeptide has the stuttering rate as described herein, an on-chip stuttering assay as described in Example 4 is used. For the purposes of determining whether a polypeptide has the procession rate as described herein, an on-chip Kₑₓₜ assay as described in Example 4 is used. In an embodiment, the polypeptide has a stuttering rate of less than 10% on a PCluc 6Kb plasmid with a HEG adapter. In another embodiment, the polypeptide has a stuttering rate of less than 7% on a E. coli genome (the E. coli 6Kb fractionated library was derived from E. coli, K12 strain, MG1655 sub-strain). In another embodiment, the polypeptide has a procession rate of greater than 4000 nucleotides. In another embodiment, the polypeptide has a procession rate of greater than 5000 nucleotides. In an embodiment, the polypeptide has a stuttering rate of less than 10% on a PCluc 6Kb plasmid and a procession length of greater than 4000 nucleotides. In another embodiment, the polypeptide has a stuttering rate of less than 7% on a E. coli genome and a procession length of greater than 4000 nucleotides. In an embodiment, the polypeptide has a stuttering rate of less than 10% on a plasmid and a procession length of greater than 5000 nucleotides. In another embodiment, the polypeptide has a stuttering rate of less than 7% on a E. coli genome and a procession length of greater than 5000 nucleotides. Exemplary substitutions at these positions relative to SEQ ID NO: 2 include N298L, L538R, P542A, I570H/T/W/R/N/G, N574L, E633 W/F, S636F, E639K, and K655G.

In a further embodiment, the substitution(s) is/are selected from the group consisting of G12 W/Y/F, K114 W/F/I, L117L/F/P, N194F/W/V, M232 W/G/R, N298L, G313E/Q/L, A451F/W/Y, K490 W/F/Y, L538R, P542A, Q565Y/I/V, I570H/T/W/R/N/G, N574L, Q590P/V/Y, E633 W/F, S636F, E639K, K655G, and D681G/N/H.

In another further embodiment, the substitution(s) is/are selected from the group consisting of G12 W/Y/F, K114 W/F/I, L117L/F/P, N194F/W/V, M232 W/G/R, N298L, G313E/Q/L, A451F/W/Y, K490 W/F/Y, L538R, P542A, Q565Y/I/V, I570H/T/W/R/N/G, N574L, Q590P/V/Y, E633 W/F, S636F, E639K, K655G, and D681G/N/H.

In a further embodiment, the substitution(s) is/are selected from the group consisting of G12 W, G12Y, G12F, A451F, A451 W, N194 W, E5651, E590Y, and D681H.

In a further embodiment, the substitution(s) is/are selected from the group consisting of A451F, N194 W, and D681H.

In a further embodiment, the substitution(s) is/are selected from the group consisting of N298L, L538R, P542A, I570H/T/W/R/N/G, N574L, E633 W/F, S636F, E639K, and K655G.

In a further embodiment, the substitution(s) is/are selected from the group consisting of I570H, N574L, S636F, L538R, and E633F.

In a further embodiment, at least one substitution is selected from the group consisting of G12 W/Y/F, K114 W/F/I, L117L/F/P, N194F/W/V, M232 W/G/R, G313E/Q/L, A451F/W/Y, K490 W/F/Y, Q565Y/I/V, Q590P/V/Y, and D681G/N/H; and at least one substitution is selected from the group consisting of N298L, L538R, P542A, I570H/T/W/R/N/G, N574L, E633 W/F, S636F, E639K, and K655G.

In a further embodiment, at least one substitution is selected from the group consisting of G12 W/Y/F, K114 W/F/I, L117L/F/P, N194F/W/V, M232 W/G/R, G313E/Q/L, A451F/W/Y, K490 W/F/Y, Q565Y/I/V, Q590P/V/Y, and D681G/N/H; and at least one substitution is selected from the group consisting of I570H, N574L, S636F, L538R, and E633F.

In a further embodiment, at least one substitution is selected from the group consisting of G12 W, G12Y, G12F, A451F, A451 W, N194 W, E5651, E590Y, and D681H; and at least one substitution is selected from the group consisting of N298L, L538R, P542A, I570H/T/W/R/N/G, N574L, E633 W/F, S636F, E639K, and K655G.

In a further embodiment, at least one substitution is selected from the group consisting of G12 W, G12Y, G12F, A451F, A451 W, N194 W, E5651, E590Y, and D681H; and at least one substitution is selected from the group consisting of I570H, N574L, S636F, L538R, and E633F.

In a further embodiment, at least one substitution is selected from the group consisting of A451F, N194 W, and D681H; and at least one substitution is selected from the group consisting of N298L, L538R, P542A, I570H/T/W/R/N/G, N574L, E633 W/F, S636F, E639K, and K655G.

In a further embodiment, at least one substitution is selected from the group consisting of A451F, N194 W, and D681H; and at least one substitution is selected from the group consisting of 1570H, N574L, S636F, L538R, and E633F.

Non-limiting examples of polypeptides include: Pol6-2268 (SEQ ID NO: 14), Pol6-2269 (SEQ ID NO: 15), Pol6-2270 (SEQ ID NO: 16), Pol6-2271 (SEQ ID NO: 5), Pol6-2272 (SEQ ID NO: 17), Pol6-2273 (SEQ ID NO: 18), Pol6-2274 (SEQ ID NO: 19), Pol6-2275 (SEQ ID NO: 20), Pol6-2276 (SEQ ID NO: 21), Pol6-2564 (SEQ ID NO: 22), Pol6-2565 (SEQ ID NO: 23), Pol6-2566 (SEQ ID NO: 24), Pol6-2567 (SEQ ID NO: 25), Pol6-2568 (SEQ ID NO: 26), Pol6-2569 (SEQ ID NO: 27), Pol6-2570 (SEQ ID NO: 28), Pol6-2571 (SEQ ID NO: 29), Pol6-2572 (SEQ ID NO: 30), Pol6-2573 (SEQ ID NO: 31), Pol6-2574 (SEQ ID NO: 32), Pol6-2575 (SEQ ID NO: 33), Pol6-2576 (SEQ ID NO: 34), Pol6-2577 (SEQ ID NO: 35), Pol6-2578 (SEQ ID NO: 36), Pol6-2579 (SEQ ID NO: 37), Pol6-2580 (SEQ ID NO: 38), Pol6-2581 (SEQ ID NO: 39), Pol6-2582 (SEQ ID NO: 40), Pol6-2583 (SEQ ID NO: 41), Pol6-2584 (SEQ ID NO: 42), Pol6-2585 (SEQ ID NO: 43), Pol6-2586 (SEQ ID NO: 44), Pol6-2587 (SEQ ID NO: 45), Pol6-2588 (SEQ ID NO: 46), Pol6-2589 (SEQ ID NO: 47), Pol6-2590 (SEQ ID NO: 48), and Pol6-2591 (SEQ ID NO: 49).

In an embodiment, the isolated polypeptide comprises, consists essentially of, or consists of, amino acid residues 11-739 of any of SEQ ID NO: 5 and 14-49. In another embodiment, the isolated polypeptide comprises, consists essentially of, or consists of, amino acid residues 11-739 of any of SEQ ID NO: 5 and 14-49. SEQ ID NO: 5 and 14-49 are described in Table 1:

TABLE 1

| | | | Substitution relative |
|---|---|---|---|
| ID | SEQ ID NO | Parental | to parental |
| Pol6-2268 | 14 | P1743 | G12W |
| Pol6-2269 | 15 | P1743 | A451F |
| Pol6-2270 | 16 | P1743 | G12Y |
| Pol6-2271 | 5 | P1743 | N194W |
| Pol6-2272 | 17 | P1743 | A451W |
| Pol6-2273 | 18 | P1743 | E565I |
| Pol6-2274 | 19 | P1743 | G12F |
| Pol6-2275 | 20 | P1743 | E590Y |
| Pol6-2276 | 21 | P1743 | D681H |
| Pol6-2564 | 22 | P2271 | N298L |
| Pol6-2565 | 23 | P2271 | I570H |
| Pol6-2566 | 24 | P2271 | I570T |
| Pol6-2567 | 25 | P2271 | I570G |
| Pol6-2568 | 26 | P2271 | E633W |
| Pol6-2569 | 27 | P2271 | K655G |
| Pol6-2570 | 28 | P2271 | N574L |
| Pol6-2571 | 29 | P2271 | S636F |
| Pol6-2572 | 30 | P2546 | N298L |
| Pol6-2573 | 31 | P2546 | L538R |
| Pol6-2574 | 32 | P2546 | I570H |
| Pol6-2575 | 33 | P2546 | I570T |
| Pol6-2576 | 34 | P2546 | I570G |
| Pol6-2577 | 35 | P2546 | N574L |
| Pol6-2578 | 36 | P2546 | E633W |
| Pol6-2579 | 37 | P2546 | E633F |
| Pol6-2580 | 38 | P2546 | S636F |
| Pol6-2581 | 39 | P2546 | E639K |
| Pol6-2582 | 40 | P2546 | K655G |
| Pol6-2583 | 41 | P2303 | N298L |
| Pol6-2584 | 42 | P2303 | E633F |
| Pol6-2585 | 43 | P2094 | N298L |
| Pol6-2586 | 44 | P2094 | L538R |
| Pol6-2587 | 45 | P2094 | I570H |
| Pol6-2588 | 46 | P2094 | I570G |
| Pol6-2589 | 47 | P2094 | E633F |
| Pol6-2590 | 48 | P2094 | E639K |
| Pol6-2591 | 49 | P2094 | K655G |

The polypeptides herein may further include other components that would be helpful in making and using them as polymerases in a nanopore-base sequencing system. For example, the polypeptide may include components useful in attaching the polypeptide to a biological nanopore. Examples include SpyTag/SpyCatcher peptide system (Zakeri et al. PNAS 109: E690-E697 2012), native chemical ligation system (Thapa et al., Molecules 19:14461-14483 2014), sortase system (Wu and Guo, J Carbohydr Chem 31:48-66 2012; Heck et al., Appl Microbiol Biotechnol 97:461-475 2013)), transglutaminase systems (Dennler et al., Bioconjug Chem 25:569 578 2014), formylglycine linkage systems (Rashidian et al., Bio conjug Chem 24:1277-1294 2013), Click chemistry attachment systems, or other chemical ligation techniques known in the art.

### Nanopore Assembly and Insertion

The methods described herein can use a nanopore having a polymerase as disclosed herein attached to the nanopore. In some cases, it is desirable to have one and only one polymerase per nanopore (e.g., so that only one nucleic acid molecule is sequenced at each nanopore). However, many nanopores, including, e.g., alpha-hemolysin (aHL), can be multimeric proteins having a plurality of subunits (e.g., 7 subunits for aHL). The subunits can be identical copies of the same polypeptide. Provided herein are multimeric proteins (e.g., nanopores) having a defined ratio of modified subunits (e.g., a-HL variants) to un-modified subunits (e.g., a-HL). Also provided herein are methods for producing multimeric proteins (e.g., nanopores) having a defined ratio of modified subunits to un-modified subunits.

With reference to FIG. 27 of WO2014/074727 (Genia Technologies, Inc.), a method for assembling a protein having a plurality of subunits comprises providing a plurality of first subunits 2705 and providing a plurality of second subunits 2710, where the second subunits are modified when compared with the first subunits. In some cases, the first subunits are wild-type (e.g., purified from native sources or produced recombinantly). The second subunits can be modified in any suitable way. In some cases, the second subunits have a protein (e.g., a polymerase) attached (e.g., as a fusion protein).

The modified subunits can comprise a chemically reactive moiety (e.g., an azide or an alkyne group suitable for forming a linkage). In some cases, the method further comprises performing a reaction (e.g., a Click chemistry cycloaddition) to attach an entity (e.g., a polymerase) to the chemically reactive moiety.

The method can further comprise contacting the first subunits with the second subunits 2715 in a first ratio to form a plurality of proteins 2720 having the first subunits and the second subunits. For example, one part modified aHL subunits having a reactive group suitable for attaching a polymerase can be mixed with six parts wild-type aHL subunits (i.e., with the first ratio being 1:6). The plurality of proteins can have a plurality of ratios of the first subunits to the second subunits. For example, the mixed subunits can form several nanopores having a distribution of stoichiometries of modified to un-modified subunits (e.g., 1:6, 2:5, 3:4).

In some cases, the proteins are formed by simply mixing the subunits. In the case of aHL nanopores for example, a detergent (e.g., deoxycholic acid) can trigger the aHL monomer to adopt the pore conformation. The nanopores can also be formed using a lipid (e.g., 1,2-diphytanoyl-sn-glycero-3-phosphocholine (DPhPC) or 1,2-di-O-phytanyl-sn-glycero-3-phosphocholine (DoPhPC)) and moderate temperature (e.g., less than about 100° C.). In some cases, mixing DPhPC with a buffer solution creates large multi-lamellar vesicles (LMV), and adding aHL subunits to this solution and incubating the mixture at 40° C. for 30 minutes results in pore formation.

If two different types of subunits are used (e.g., the natural wild type protein and a second aHL monomer which can contain a single point mutation), the resulting proteins can have a mixed stoichiometry (e.g., of the wild type and mutant proteins). The stoichiometry of these proteins can follow a formula which is dependent upon the ratio of the concentrations of the two proteins used in the pore forming reaction.

This formula is as follows:

100Pₘ=100(n!/m!(n−m)!)·fₘᵤₜ^{m}·f_{wt}^{n˜m}, where
Pₘ=probability of a pore having m number of mutant subunits
n=total number of subunits (e.g., 7 for aHL)
m=number of “mutant” subunits
fₘᵤₜ=fraction or ratio of mutant subunits mixed together
f_{wt}=fraction or ratio of wild-type subunits mixed together

The method can further comprise fractionating the plurality of proteins to enrich proteins that have a second ratio of the first subunits to the second subunits 2725. For example, nanopore proteins can be isolated that have one and only one modified subunit (e.g., a second ratio of 1:6). However, any second ratio is suitable. A distribution of second ratios can also be fractionated such as enriching proteins that have either one or two modified subunits. The total number of subunits forming the protein is not always 7 (e.g., a different nanopore can be used or an alpha-hemolysin nanopore can form having six subunits) as depicted in FIG. 27 of WO2014/074727. In some cases, proteins having only one modified subunit are enriched. In such cases, the second ratio is 1 second subunit per (n−1) first subunits where n is the number of subunits comprising the protein.

The first ratio can be the same as the second ratio, however this is not required. In some cases, proteins having mutated monomers can form less efficiently than those not having mutated subunits. If this is the case, the first ratio can be greater than the second ratio (e.g., if a second ratio of 1 mutated to 6 non-mutated subunits are desired in a nanopore, forming a suitable number of 1:6 proteins may require mixing the subunits at a ratio greater than 1:6).

Proteins having different second ratios of subunits can behave differently (e.g., have different retention times) in a separation. In some cases, the proteins are fractionated using chromatography, such as ion exchange chromatography or affinity chromatography. Since the first and second subunits can be identical apart from the modification, the number of modifications on the protein can serve as a basis for separation. In some cases, either the first or second subunits have a purification tag (e.g., in addition to the modification) to allow or improve the efficiency of the fractionation. In some cases, a poly-histidine tag (His-tag), a streptavidin tag (Strep-tag), or other peptide tag is used. In some instances, the first and second subunits each comprise different tags and the fractionation step fractionates on the basis of each tag. In the case of a His-tag, a charge is created on the tag at low pH (Histidine residues become positively charged below the pKa of the side chain). With a significant difference in charge on one of the aHL molecules compared to the others, ion exchange chromatography can be used to separate the oligomers which have 0, 1, 2, 3, 4, 5, 6, or 7 of the “charge-tagged” aHL subunits. In principle, this charge tag can be a string of any amino acids which carry a uniform charge. FIG. 28 and FIG. 29 show examples of fractionation of nanopores based on a His-tag. FIG. 28 shows a plot of ultraviolet absorbance at 280 nanometers, ultraviolet absorbance at 260 nanometers, and conductivity. The peaks correspond to nanopores with various ratios of modified and unmodified subunits. FIG. 29 of WO2014/074727 shows fractionation of aHL nanopores and mutants thereof using both His-tag and Strep-tags.

In some cases, an entity (e.g., a polymerase as disclosed herein) is attached to the protein following fractionation. The protein can be a nanopore and the entity can be a polymerase. In some instances, the method further comprises inserting the proteins having the second ratio subunits into a bilayer.

In some situations, a nanopore can comprise a plurality of subunits. A polymerase can be attached to one of the subunits and at least one and less than all of the subunits comprise a first purification tag. In some examples, the nanopore is alpha-hemolysin or a variant thereof. In some instances, all of the subunits comprise a first purification tag or a second purification tag. The first purification tag can be a poly-histidine tag (e.g., on the subunit having the polymerase attached).

### Polymerase Attached to Nanopore

In some cases, a polymerase (e.g., the polypeptides disclosed herein) is attached to and/or is located in proximity to the nanopore. The polymerase can be attached to the nanopore before or after the nanopore is incorporated into the membrane. In some instances, the nanopore and polymerase are a fusion protein (i.e., single polypeptide chain).

The polymerase can be attached to the nanopore in any suitable way. In some cases, the polymerase is attached to the nanopore (e.g., hemolysin) protein monomer and then the full nanopore heptamer is assembled (e.g., in a ratio of one monomer with an attached polymerase to 6 nanopore (e.g., hemolysin) monomers without an attached polymerase). The nanopore heptamer can then be inserted into the membrane.

Another method for attaching a polymerase to a nanopore involves attaching a linker molecule to a hemolysin monomer or mutating a hemolysin monomer to have an attachment site and then assembling the full nanopore heptamer (e.g., at a ratio of one monomer with linker and/or attachment site to 6 hemolysin monomers with no linker and/or attachment site). A polymerase can then be attached to the attachment site or attachment linker (e.g., in bulk, before inserting into the membrane). The polymerase can also be attached to the attachment site or attachment linker after the (e.g., heptamer) nanopore is formed in the membrane. In some cases, a plurality of nanopore-polymerase pairs are inserted into a plurality of membranes (e.g., disposed over the wells and/or electrodes) of the biochip. In some instances, the attachment of the polymerase to the nanopore complex occurs on the biochip above each electrode.

The polymerase can be attached to the nanopore with any suitable chemistry (e.g., covalent bond and/or linker). In some cases, the polymerase is attached to the nanopore with molecular staples. In some instances, molecular staples comprise three amino acid sequences (denoted linkers A, B and C). Linker A can extend from a hemolysin monomer, Linker B can extend from the polymerase, and Linker C then can bind Linkers A and B (e.g., by wrapping around both Linkers A and B) and thus the polymerase to the nanopore. Linker C can also be constructed to be part of Linker A or Linker B, thus reducing the number of linker molecules.

In some instances, the polymerase is linked to the nanopore using Solulink™ chemistry. Solulink™ can be a reaction between HyNic (6-hydrazino-nicotinic acid, an aromatic hydrazine) and 4FB (4-formylbenzoate, an aromatic aldehyde). In some instances, the polymerase is linked to the nanopore using Click chemistry (available from LifeTechnologies for example). In some cases, zinc finger mutations are introduced into the hemolysin molecule and then a molecule is used (e.g., a DNA intermediate molecule) to link the polymerase to the zinc finger sites on the hemolysin.

Other linkers that may find use in attaching the polymerase to a nanopore are direct genetic linkage, transglutaminase mediated linking, sortase mediated linking, and chemical linking through cysteine modifications.

### Apparatus Set-Up

The nanopore may be formed or otherwise embedded in a membrane disposed adjacent to a sensing electrode of a sensing circuit, such as an integrated circuit. The integrated circuit may be an application specific integrated circuit (ASIC). In some examples, the integrated circuit is a field effect transistor or a complementary metal-oxide semiconductor (CMOS). The sensing circuit may be situated in a chip or other device having the nanopore, or off of the chip or device, such as in an off-chip configuration. The semiconductor can be any semiconductor, including, without limitation, Group IV (e.g., silicon) and Group III-V semiconductors (e.g., gallium arsenide). See, for example, US20180306746A1, for the apparatus and device set-up for sensing a nucleotide or tag.

Pore based sensors (e.g., biochips) can be used for electro-interrogation of single molecules. A pore based sensor can include a nanopore of the present disclosure formed in a membrane that is disposed adjacent or in proximity to a sensing electrode. The sensor can include a counter electrode. The membrane includes a trans side (i.e., side facing the sensing electrode) and a cis side (i.e., side facing the counter electrode).

In the experimental disclosure which follows, the following abbreviations apply: eq (equivalents); M (Molar); μM (micromolar); N (Normal); mol (moles); mmol (millimoles); μmol (micromoles); nmol (nanomoles); g (grams); mg (milligrams); kg (kilograms); μg (micrograms); L (liters); ml (milliliters); μl (microliters); cm (centimeters); mm (millimeters); μm (micrometers); nm (nanometers); ° C. (degrees Centigrade); h (hours); min (minutes); sec (seconds); msec (milliseconds).

### EXAMPLES

The present disclosure is described in further explained in the following examples which are not in any way intended to limit the scope of the disclosure as claimed. The attached Figures are meant to be considered as integral parts of the specification and description of the disclosure. All references cited are herein specifically incorporated by reference for all that is described therein. The following examples are offered to illustrate, but not to limit the claimed disclosure.

### Example 1

### Molecular Modeling, Selection of Variants, and Confirmation of Polymerase Activity

A previously isolated Pol6 variant (Pol6-1743, SEQ ID NO: 3 was selected for targeted mutagenesis to improve thermal stability. Pol6-1743 comprises the following substitutions relative to SEQ ID NO: 2: T529M, S366A, A547F, N545L, Y225L, D657R, K561G, K541R, T544A, S692Y, V299R, E680Q, Y242A, E585K, V164K, F528Y, N635R, E534R, P523F, Q221K, and K682Y.

Molecular modeling was used to calculate an accurate structural model of Pol6-1743 and molecular dynamics employed to interrogate its thermodynamic profile and determine likely functional regions. Intra-protein residue interaction mapping coupled with in silico whole protein deep mutational scanning was utilized to evaluate putative mutants that could stabilize the thermodynamics of surface exposed non-functional flexible regions. Without being bound by theory, mutations were selected that could introduce interactions with multiple distal residues in the local 3-dimensional neighborhood through either hydrogen bond, hydrophobic, pi-cation, pi-stacking or salt-bridge interactions which are hypothesized to make the protein as a whole less dynamic and more compact by nature more thermostable.

A total of 11 loci were identified using this approach and 3 mutant residues selected at each position based on the number of relative putative stabilizing interactions introduced into the local neighborhood to represent strong, moderate and weak stabilizing forces. The specific 33 mutations are as follows: G12 W/Y/F, K114 W/F/I, L117L/F/P, N194F/W/V, M232 W/G/R, G313E/Q/L, A451F/W/Y, K490 W/F/Y, Q565Y/I/V, Q590P/V/Y, and D681G/N/H. Single substitution variants relative to Pol6-1743 were generated by site-directed mutagenesis and expressed according to standard methods in the art.

A fluorescent displacement assay was performed to evaluate the procession rate (Kₑₓₜ) of the variant Pol6 DNA polymerases relative to Pol6-1743. In brief, a hairpin template with a fluorophore was annealed to a primer with a quencher molecule. Upon extension of the hairpin template by the polymerase, the quencher primer was displaced and the fluorescent signal was measured. The change in fluorescence over time was measured in real time and used to determine Kₑₓₜ. 30 of 33 variants were determined to be active and the kinetics were not significantly affected by the substitutions.

### Example 2

### Off-Chip Determination of Thermostability

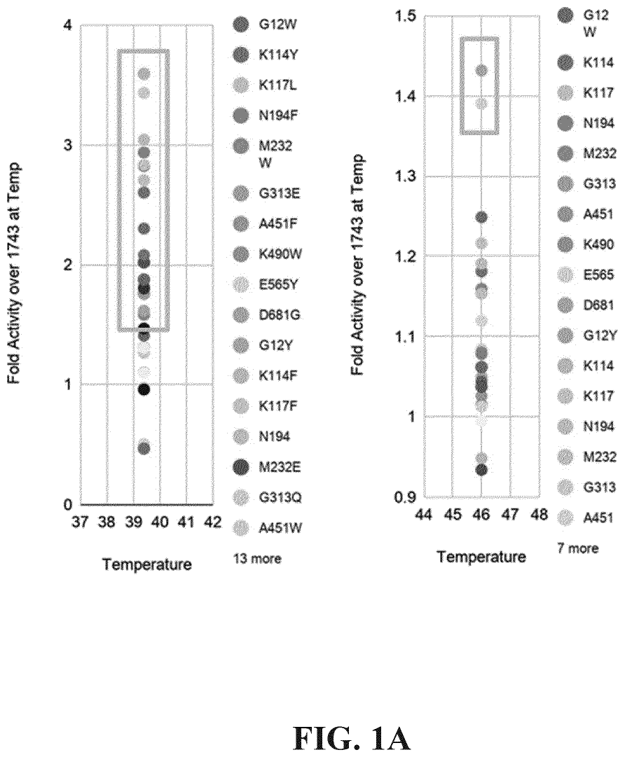

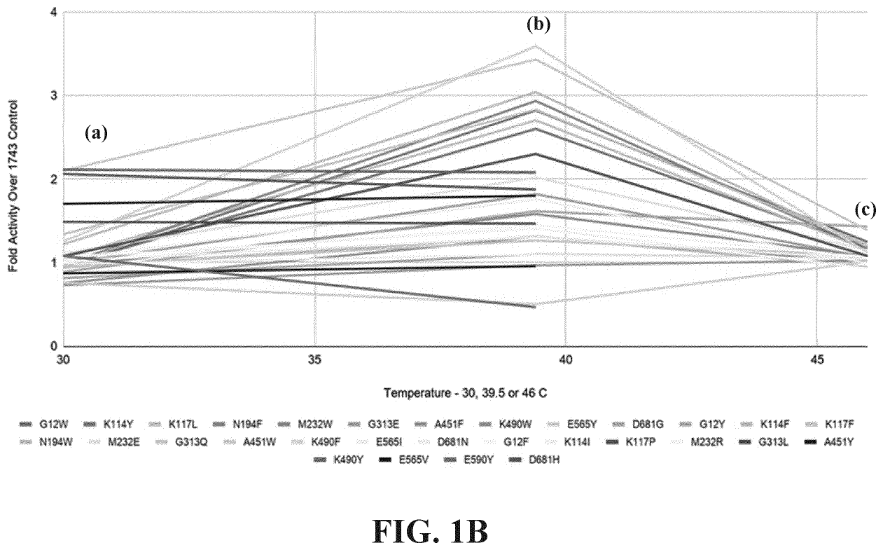

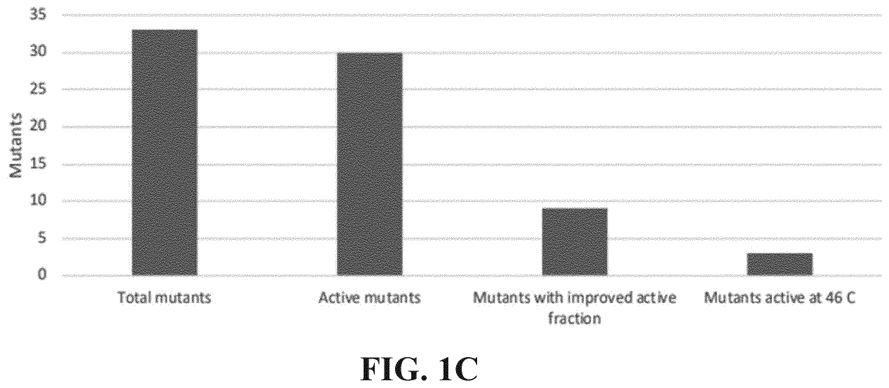

The 30 active variants were then subjected to an active fraction assay using a temperature range of 37 to 48° C. An “active fraction assay” measures the amplitude of extension in template-dependent polymerase reaction. Higher amplitude indicates that a higher percentage of polymerase in the reaction mix is active, thereby indicating higher stability of the complex. Results are shown at FIG. 1A-1C.

As illustrated at FIG. 1A, 15 of the 30 active variants showed at least a 1.5-fold amplitude in extension (relative to Pol6-1743) at 39 to 40° C. (box in left graph), while 2 additional variants showed retention of activity at temperatures up to 46° C. (box in right graph). As illustrated in FIG. 1B, three distinct improvements in stability can be observed: (a) 5 variants had at least 1.5-fold higher active fraction than Pol6-1743 at 30° C.; (b) 15 variants had at least 1.5-fold higher active fraction than Pol6-1743 at between 39 and 40° C.; and (c) 2 variants retain greater than 1-fold higher active fraction than Pol6-1743 at 40° C. As summarized at FIG. 1C, 30/33 variants retain an active fraction, of which 9 demonstrated improved active fraction, and 3 showed activity after heating to 46° C. The data demonstrate improved thermostability of these variants relative to Pol6-1743.

### Example 3

TABLE 2
On-Chip Determination of Kinetics and Thermostability
The following were evaluated for on-chip stability relative to Pol6-1743:

| | | Substitution relative |
|---|---|---|
| ID | SEQ ID NO | to Pol6-1743 |
| Pol6-2268 | 14 | G12W |
| Pol6-2269 | 15 | A451F |
| Pol6-2270 | 16 | G12Y |
| Pol6-2271 | 5 | N194W |
| Pol6-2272 | 17 | A451W |
| Pol6-2273 | 18 | E565I |
| Pol6-2274 | 19 | G12F |
| Pol6-2275 | 20 | E590Y |
| Pol6-2276 | 21 | D681H |

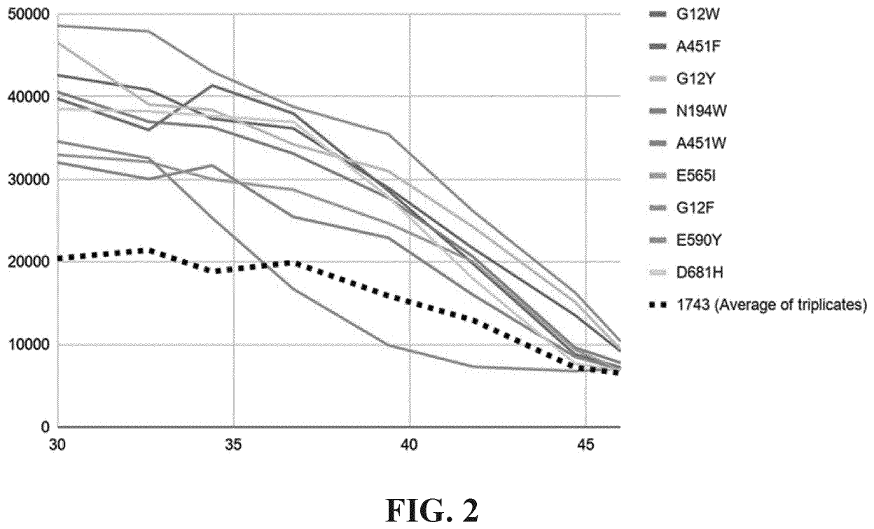

Results are shown at FIG. 2. All 9 demonstrated improved stability relative to Pol6-1743 at temperatures below 35° C. Above 35° C., only Pol6-2268 showed significant on-chip performance decrease relative to Pol6.

Pol6-2269 (A451F), Pol6-2271 (N194 W) and Pol6-2276 (D681H) were picked for characterization of on-chip kinetics. A chip was set up essentially as described in US20180306746A1, using the following conditions:

TABLE 3
SEQUENCING CONDITIONS

| Trans Buffer | 510 mM KOAc, 18 mM MgOAc, 15 mM LiOAc, 5 mM KCl, 1% | |
|---|---|---|
| | Trehalose, 500 μM EDTA, 0.09% ProClin 300, 50 mM HEPES, | |
| | pH 7.8 | |
| Cis Buffer | 400 mM KOAc, 18 mM MgOAc, 15 mM LiOAc, 3 μM PEG- | |
| | tagged dN6OP, 5 mM TCEP, 500 uM EDTA, 0.09% ProClin 300, | |
| | 50 mM HEPES, pH 7.8 | |
| Pore | 6-monomer | SEQ ID NO: 50 |
| | 1-monomer | SEQ ID NO: 51 |

| | |
|---|---|
| Template | Barcoded 2.7 kb pUC19 insert with Dumbbell-Adaptor (~0.5 nM |
| | Loading) |
| Lipid | DPhPC in PDM20 |
| Waveform | Waveform: 1.4 kHz VPre Modulation, 180 mV p-p, 180 mV Ref |
| | Window, 1800 s, 20 C |

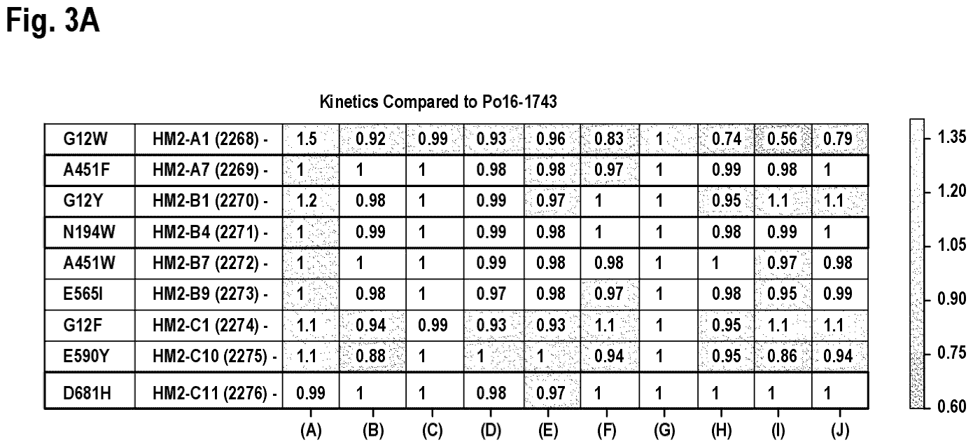

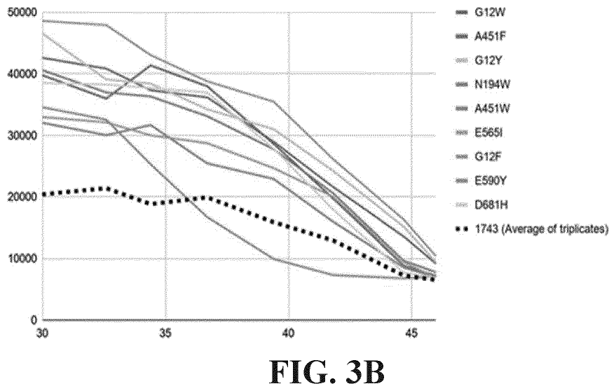

Results are shown at FIG. 3A & FIG. 3B.

FIG. 3A is a heat map comparing variant kinetics relative to Pol6-1743 for each of the following characteristics: (A) average waiting time, (B) mean dwell time, (C) mean stuttering, (D) mean stuttering rate, (E) percentage of insertions, (F) percentage of deletions, (G) accuracy, (H) procession rate, (I) procession length, and (J) sequencing lifetime. In each case, an increase in the measured characteristic relative to Pol6-1743 was considered an improvement. FIG. 3B illustrates the on-chip active fraction of each variant compared to Pol6-1743 at various temperatures between 30 and 46° C.

Given their good kinetic profiles and improved thermostability relative to Pol6-1743, Pol6-2269 (A451F) and 2271 (N194 W) were selected for further characterization. The chip was set up as described above, using the following conditions:

TABLE 4
SEQUENCING CONDITIONS

| Trans Buffer | 510 mM KOAc, 18 mM MgOAc, 15 mM LiOAc, 5 mM KCl, 1% | |
|---|---|---|
| | Trehalose, 500 μM EDTA, 0.09% ProClin 300, 50 mM HEPES, | |
| | pH 7.8 | |
| Cis Buffer | 400 mM KOAc, 18 mM MgOAc, 15 mM LiOAc, 3 μM PEG- | |
| | tagged dN6OP, 5 mM TCEP, 500 uM EDTA, 0.09% ProClin 300, | |
| | 50 mM HEPES, pH 7.8 | |
| Pore | 6-monomer | SEQ ID NO: 50 |
| | 1-monomer | SEQ ID NO: 51 |

| | |
|---|---|
| Template | T344 3 kb ID9 insert with HEG-Adaptor, P3-Short Primer, 1 nM |
| | Loading |
| Lipid | DPhPC in PDM20 |
| Waveform | Waveform: 1.4 kHz VPre Modulation, 180 mV p-p, 180 mV Ref |
| | Window, 1800 s, 20 C |

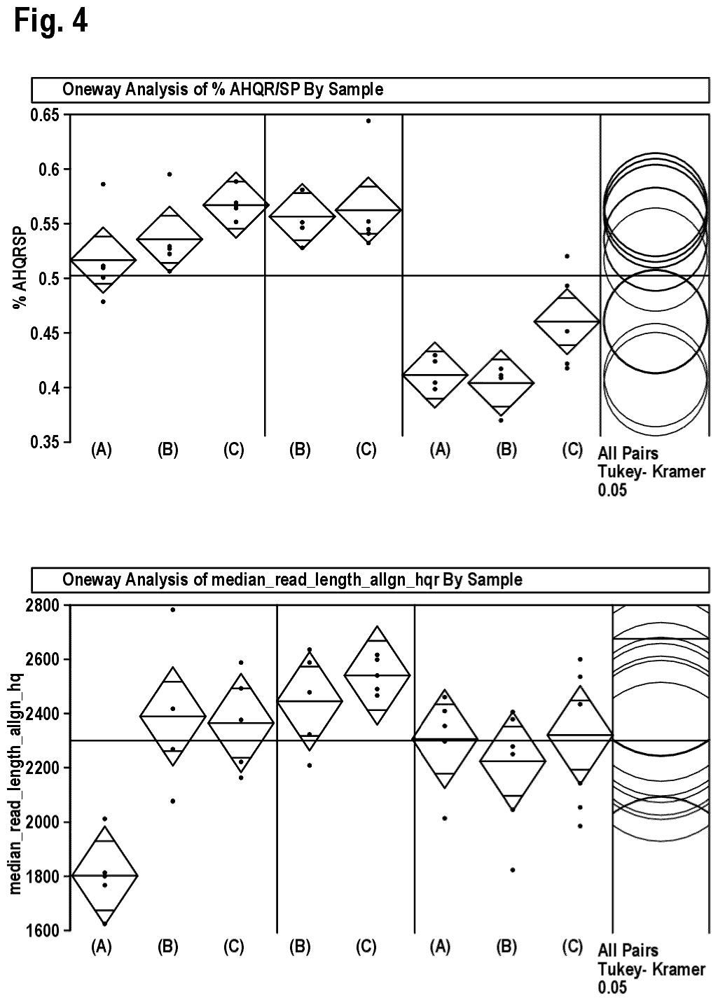

Results are shown at FIG. 4. The top graph illustrates the on-chip active fraction of Pol6-1743 (A), Pol6-2269 (B), and Pol6-2271 (C) at each of 36, 38, and 40° C. The bottom graph illustrates the median read length for the same polymerases at the same temperatures. The variant polymerases showed a 2-5% improvement in on-chip active fraction (% oAQTIR/SP) at 36 and 38° C. Also, Pol6-2269 and Pol6-2271 had increased median read length significantly compared to 1743 at 36° C., 38° C. and 40° C.

### Example 3

### Modification of Variants to Improve Kinetics

Further substitutions (diversity) to reduce insertions (stutters) were selected by mining a database of historical Pol6 mutants. A total of 15 additional substitutions were identified: N298L, L538R, P542A, J570H/IT/W/R/N/G, N574L, E633 W/F, S636F, E639K, and K655G. 4 different Pol6 backbones (Pol6-2094 (SEQ ID NO: 4), Pol6-2271 (SEQ TD NO: 5), Pol6-2303 (SEQ TD NO: 6), and Pol6-2546 (SEQ ID NO: 7)) were selected for improvement, and 48 of a total of 60 possible single substitution variants were generated and tested for activity by Kₑₓₜ assay as previously described. 26 of the 48 generated variants were determined to be active.

The active polymerases were tested for Kₑₓₜ relative to the parental backbone. Results are shown below:

TABLE 5

| ID | SEQ ID NO | Parental | Substitution | Fold Kₑₓₜ |
|---|---|---|---|---|
| Pol6-2564 | 22 | P2271 | N298L | 1.00 |
| Pol6-2565 | 23 | P2271 | I570H | 1.09 |
| Pol6-2566 | 24 | P2271 | I570T | 1.05 |
| Pol6-2567 | 25 | P2271 | I570G | 1.17 |
| Pol6-2568 | 26 | P2271 | E633W | 1.28 |
| Pol6-2569 | 27 | P2271 | K655G | 1.16 |
| Pol6-2570 | 28 | P2271 | N574L | 1.10 |
| Pol6-2571 | 29 | P2271 | S636F | 1.23 |
| Pol6-2572 | 30 | P2546 | N298L | 0.86 |
| Pol6-2573 | 31 | P2546 | L538R | 1.20 |
| Pol6-2574 | 32 | P2546 | I570H | 1.10 |
| Pol6-2575 | 33 | P2546 | I570T | 1.00 |
| Pol6-2576 | 34 | P2546 | I570G | 1.15 |
| Pol6-2577 | 35 | P2546 | N574L | 1.03 |
| Pol6-2578 | 36 | P2546 | E633W | 1.29 |
| Pol6-2579 | 37 | P2546 | E633F | 1.18 |
| Pol6-2580 | 38 | P2546 | S636F | 0.97 |
| Pol6-2581 | 39 | P2546 | E639K | 1.08 |
| Pol6-2582 | 40 | P2546 | K655G | 1.14 |
| Pol6-2583 | 41 | P2303 | N298L | 1.15 |
| Pol6-2584 | 42 | P2303 | E633F | 1.04 |
| Pol6-2585 | 43 | P2094 | N298L | 0.68 |
| Pol6-2586 | 44 | P2094 | L538R | 0.87 |
| Pol6-2587 | 45 | P2094 | I570H | 0.73 |
| Pol6-2588 | 46 | P2094 | I570G | 0.79 |
| Pol6-2589 | 47 | P2094 | E633F | 0.88 |
| Pol6-2590 | 48 | P2094 | E639K | 0.86 |
| Pol6-2591 | 49 | P2094 | K655G | 0.71 |

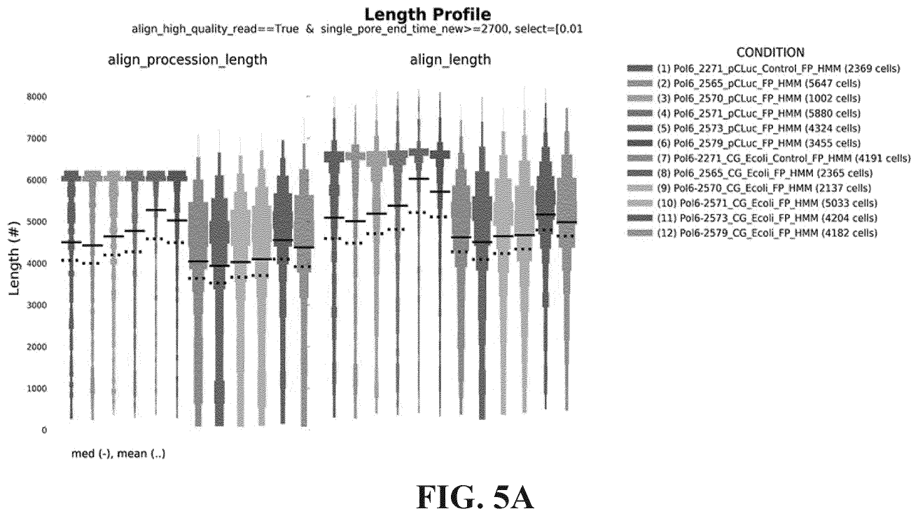

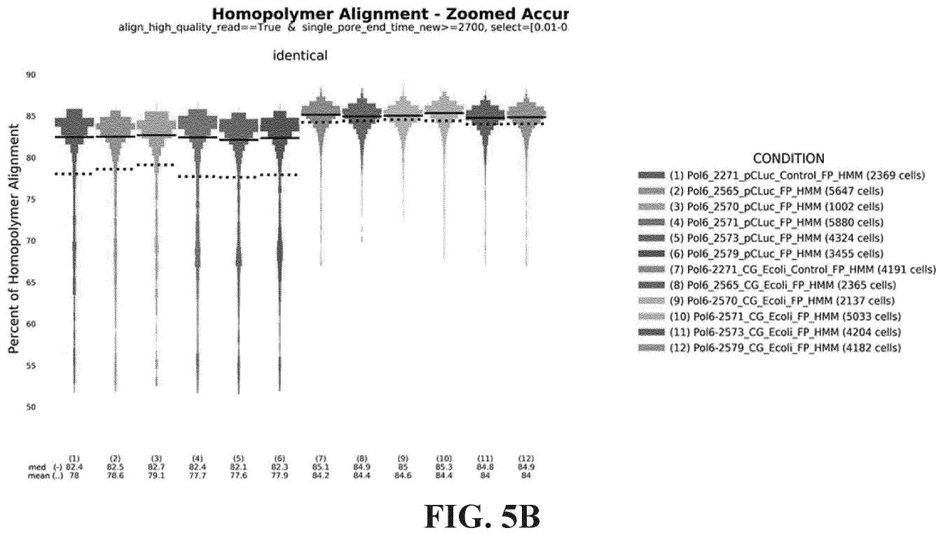

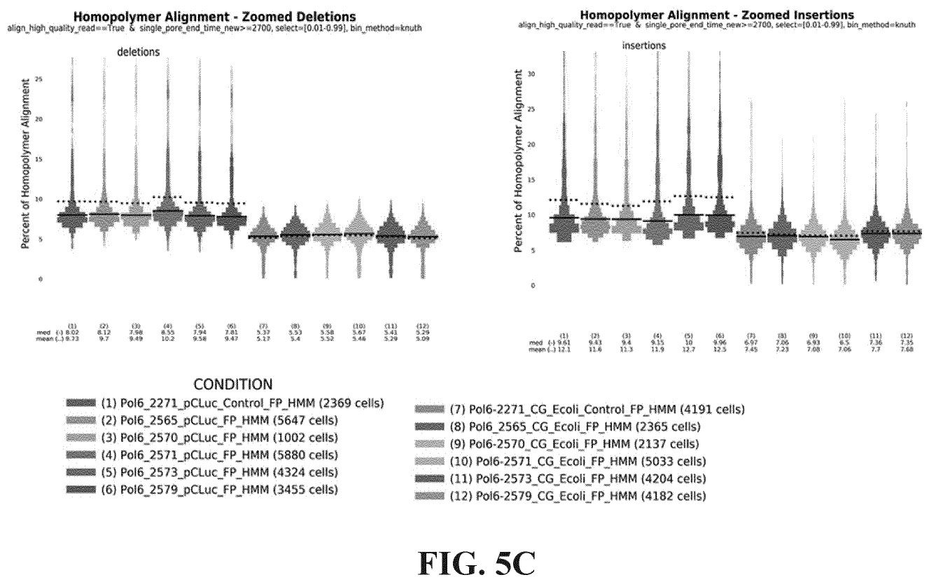

5 variants (Pol6-2565, Pol6-2570, Pol6-2571, Pol6-2573 and Pol6-2579) were down selected based on on-chip data and re-tested again on-chip with plasmid and E. Coli genomic templates for stutter. Results are shown at FIG. 5A (Length Profile), FIG. 5B (Accuracy), and FIG. 5C (Percent deletions (left graph) and insertions (right graph)). All variants except 2565 showed significant improvement in length with neutral or minimal effect on accuracy, deletions and insertions.

1. An isolated polypeptide having a DNA polymerase activity, the isolated polypeptide comprising an amino acid sequence having at least 75% sequence identity to amino acids 11-739 of SEQ ID NO: 2 and comprising at least one amino acid substitution relative to SEQ ID NO: 2 at a position selected from the group consisting of G12, K114, L117, N194, M232, N298, G313, A451, K490, L538, P542, Q565, 1570, N574L, Q590, E633, S636, E639, K655, and D681.

2. The isolated polypeptide of claim 1, wherein the at least one substitution is selected from the group consisting of G12W/Y/F, K114W/F/I, L117L/F/P, N194F/W/V, M232W/G/R, G313E/Q/L, A451F/W/Y, K490W/F/Y, Q565Y/I/V, Q590P/V/Y, and D681G/N/H.

3. The isolated polypeptide of any one of the preceding claims, wherein the at least one substitution is selected from the group consisting of G12W, G12Y, G12F, A451F, A451W, N194W, E5651, E590Y, and D681H.

4. The isolated polypeptide of any one of the preceding claims, wherein the at least one substitution is selected from the group consisting of A451F, N194W, D681H.

5. The isolated polypeptide of any of any one of the preceding claims, wherein said isolated polypeptide has an improved thermostability relative to Pol6-1743 at a temperature in the range of 37° C. to 47° C.

6. The isolated polypeptide of any one of the preceding claims, wherein the isolated polypeptide has at least 1.5-fold greater thermostability of DNA polymerase activity relative to Pol6-1743 at 36° C., 38° C., 40° C., and/or 46° C.

7. The isolated polypeptide of any of any one of the preceding claims, wherein said amino acid sequence further comprises at least one substitution selected from the group consisting of N298L, L538R, P542A, I570H/T/W/R/N/G, N574L, E633 W/F, S636F, E639K, and K655G.

8. The isolated polypeptide of any one of the preceding claims, wherein the substitutions are selected from the group consisting of I570H, N574L, S636F, L538R, and E633F.

9. The isolated polypeptide of any one of the preceding claims, wherein said polypeptide comprises at least one substitution selected from the group consisting of G12W/Y/F, K114W/F/I, L117L/F/P, N194F/W/V, M232W/G/R, G313E/Q/L, A451F/W/Y, K490W/F/Y, Q565Y/I/V, Q590P/V/Y, and D681G/N/H; and at least one substitution is selected from the group consisting of N298L, L538R, P542A, I570H/T/W/R/N/G, N574L, E633W/F, S636F, E639K, and K655G.

10. The isolated polypeptide of any one of the preceding claims, wherein at least one substitution is selected from the group consisting of G12W/Y/F, K114W/F/I, L117L/F/P, N194F/W/V, M232W/G/R, G313E/Q/L, A451F/W/Y, K490W/F/Y, Q565Y/I/V, Q590P/V/Y, and D681G/N/H; and at least one substitution is selected from the group consisting of I570H, N574L, S636F, L538R, and E633F.

11. The isolated polypeptide of any one of the preceding claims, wherein at least one substitution is selected from the group consisting of G12W, G12Y, G12F, A451F, A451W, N194W, E5651, E590Y, and D681H; and at least one substitution is selected from the group consisting of N298L, L538R, P542A, I570H/T/W/R/N/G, N574L, E633W/F, S636F, E639K, and K655G.

12. The isolated polypeptide of any one of the preceding claims, wherein at least one substitution is selected from the group consisting of G12W, G12Y, G12F, A451F, A451 W, N194 W, E5651, E590Y, and D681H; and at least one substitution is selected from the group consisting of I570H, N574L, S636F, L538R, and E633F.

13. The isolated polypeptide of any one of the preceding claims, wherein at least one substitution is selected from the group consisting of A451F, N194W, and D681H; and at least one substitution is selected from the group consisting of N298L, L538R, P542A, I570H/T/W/R/N/G, N574L, E633W/F, S636F, E639K, and K655G.

14. The isolated polypeptide of any one of the preceding claims, wherein at least one substitution is selected from the group consisting of A451F, N194W, and D681H; and at least one substitution is selected from the group consisting of I570H, N574L, S636F, L538R, and E633F.

15. A composition comprising a DNA polymerase attached to nanopore, wherein said DNA polymerase comprises the isolated polypeptide of any one of the preceding claims.

16. A biochip comprising a nanopore formed in a membrane disposed adjacent to a sensing electrode, wherein said nanopore is attached to a DNA polymerase comprising a polypeptide according to any one of claim 1-15.
